(19) United States
(12) Patent Application Publication
(10) Pub. No.: US 2013/0338114 A1
(43) Pub. Date: Dec. 19, 2013

(54) COMPOSITIONS FOR INCREASING STRENGTH, MUSCLE MASS, AND LEAN BODY MASS

(71) Applicant: Chemi Nutra, White Bear Lake, MN (US)

(72) Inventors: Lorenzo DE FERRA, Patrica (IT); Marvin A. HEUER, Orlando, FL (US); Scott L. HAGERMAN, North Oaks, MN (US); Martin PURPURA, Austin, TX (US); Ralf JAEGER, Milwaukee, WI (US)

(73) Assignee: Chemi Nutra, White Bear Lake, MN (US)

(21) Appl. No.: 13/901,423

(22) Filed: May 23, 2013

Related U.S. Application Data
(60) Provisional application No. 61/650,922, filed on May 23, 2012.
(60) Provisional application No. 61/798,809, filed on Mar. 15, 2013.

(51) Int. Cl. A61K 31/661

(52) U.S. Cl. CPC A61K 31/661
USPC 514/121

(57) ABSTRACT

Phosphatidic acid, lyso-phosphatidic acid, and/or phospholipase D can be administered to exercising mammals to increase muscle mass and strength. These actives can be administered orally to aging, bedridden or cachectic patients, as well as resistance training individuals, to improve nitrogen balance. The oral administration of phosphatidic acid as well as other actives described herein was found to increase muscle hypertrophy, strength, and lean body mass, and to decrease body fat in subjects, for example, when combined with resistance training.

### RELATED APPLICATIONS

This application claims priority under 35 U.S.C. §119(e) to U.S. Provisional Patent Application Nos. 61/650,922, filed May 23, 2012, and 61/798,809, filed Mar. 15, 2013, which applications are incorporated herein by reference.

### BACKGROUND OF THE INVENTION

Muscles are the engines that move the body. Muscles are composed of the contractile proteins myosin and actin, which together form myofibrils. Contraction occurs when actin ratchets over myosin, shortening the length of myofibrils. Like all proteins, these contractile proteins begin with the genetic response, through the ribosomal synthetic apparatus. The resulting proteins are incorporated into existing myofibrils to increase the size of a muscle or to repair any damage that occurs during contraction. The increase in size of a muscle is called muscular hypertrophy. This system requires adequate nutrition to provide amino acids to form the protein, and the pathways are controlled by various activating factors.

Muscular hypertrophy can be is achieved by exercise, especially exercise vigorous enough to reach the anaerobic threshold. Within a short time of commencing such exercise, a mammal can achieve measurable increases in muscle mass and strength. The increased demand causes the synthetic machinery to be up regulated. The activating factors that can initiate upregulation in response to demands include the “second messenger system”, which is known to include phospholipases, protein kinases and other enzymes.

During growth, pregnancy, and muscle development, the metabolism is in the anabolic phase, that is, more muscle is added than is broken down during the catabolic phase. Understanding the complexities of anabolism and catabolism and particularly, shifting the balance toward anabolism, is an ongoing and active research area.

The anabolic/catabolic balance is an important factor in disease and disease management. Muscle wasting in patients on bed rest is a common and problematic clinical issue. Patients in intensive care units often become catabolic. In many cases muscle tissue begins to deteriorate almost immediately after confinement. Astronauts become catabolic in the weightless environment of space and also begin losing muscle tissue and strength almost immediately in that environment. Even exercise is not completely sufficient to keep up with the muscle lost through catabolism the weightless environment of space. Significant loss of muscle has also been shown even in healthy, young volunteers whose leg has been immobilized a cast for only two weeks (Hespel et al. J. Physiol. 536:625-633, 2001). Extreme loss of muscle tissue leads cachexia, which is often seen in cancer, trauma and burn patients.

A shift toward catabolism may occur as a normal part of aging. Extraordinary measures are necessary to stave it off and shift the metabolism to a more anabolic state. Athletes also regularly seek to achieve a more anabolic state to enhance muscle development. In their training, especially in weight or cardiovascular training intense enough to reach the anaerobic threshold, they are regularly tearing down muscle fiber (catabolism) followed by rebuilding the fibers (anabolism). Muscle rebuilding is especially rapid during the first 90 minutes following vigorous exercise (the “anabolic window”). While daily training itself increases muscle mass and strength, the addition of certain elements, vitamins, and minerals to daily nutrition through supplementation helps increase muscle repair and growth.

Protein is the main nitrogen-containing compound in the human body. About 60%-70% of protein is found in muscle mass. A convenient measure of the anabolic/catabolic status is the nitrogen balance: the ratio between nitrogen ingested and nitrogen excreted. A positive nitrogen balance indicates net growth and an increase in muscle mass; equilibrium indicates a zero balance; while a negative nitrogen balance, if chronic, is an indication of bodily dysfunction that can lead to cachexia.

Accordingly, methods and compositions to upregulate protein synthesis, in particular the synthesis of contractile proteins, to improve the anabolic/catabolic ratio and nitrogen balance in both athletes and other persons are needed in the art.

### SUMMARY OF THE INVENTION

The invention provides compositions and methods for the administration of therapeutically effective amounts of naturally occurring, isolated compounds that are biologically active in increasing muscle mass and strength by stimulation of anabolic metabolism. The invention therefore provides for the oral use of phosphatidic acid (PA) for the enhancement of muscle mass and strength in mammals such as humans, equines and canines. The invention further provides a particularly a novel PA form from soy lecithin called PA-enriched phospholipids, and methods for the administration of PA and PA-enriched phospholipids. The methods can also be applied to reversing the muscle catabolism that leads to sarcopenia, for example, in bedridden, aging or cachectic subjects, or those in a weightless environment.

Compositions having a therapeutically effective amount of PA or PA-enriched phospholipids sufficient to affect intracellular and extracellular concentrations of PA in a mammal can shift the metabolism from a catabolic state to an anabolic state. This shift counteracts the decrease in muscle tissue that occurs with normal aging, and in more extreme cases such as bed rest, cachexia, and weightlessness. The compositions and methods can also increase exercise capacity in normal healthy mammals where increased muscle mass and strength is desired.

Accordingly, the invention provides a method for increasing skeletal muscle hypertrophy comprising orally administering to a subject an effective amount of phosphatidic acid (PA) or lysophosphatidic acid (LPA). The administration can be on a daily basis, for example, in doses of once, twice, or three times per day. The method is highly effective when the administration is continuous on a substantially daily basis for at least about two weeks, least about three weeks, at least about four week, or at least about six weeks. The methods are even more effective when the administration is substantially continuous, e.g., on a daily basis, for at least about eight weeks. The effects of the methods are further increased when the subject participates in resistance training, such as at least once, twice, or three times per week, during the phosphatidic acid or lysophosphatidic acid administration, thereby resulting in increased skeletal muscle hypertrophy in the subject, although resistance training is not always required to obtain some of the benefits of the phosphatidic acid or lysophosphatidic acid administration.

The increased skeletal muscle hypertrophy can be accompanied by an increase in skeletal muscle strength, for example, an increase in muscle hypertrophy of the rectus femoris muscles, and/or an increase in the pectoral muscles. The increased skeletal muscle hypertrophy can also be accompanied by an increase in lean body mass, for example, an increase of at least 1%, at least 2%, at least 3%, or at least 4%, such as in increase in lean body mass of about 1-4% over the course of an eight week cycle of PA or LPA administration when combined with resistance training.

The methods can be accompanied by a decrease in body fat composition, for example, a decrease of at least about 1% of a subject's body fat, a decrease of at least about 2% of a subject's body fat, a decrease of at least about 3% of a subject's body fat, a decrease of at least about 4% of a subject's body fat, a decrease of at least about 5% of a subject's body fat, a decrease of at least about 6% of a subject's body fat, a decrease of at least about 7.5% of a subject's body fat, a decrease of at least about 9% of a subject's body fat, or a decrease of about 1-10% of a subject's body fat, particularly when the administration of the PA or LPA is in combination with a resistance training regiment, and when the PA or LPA is administered for at least three weeks, at least four weeks, or at least about eight weeks.

An effective amount of phosphatidic acid or lysophosphatidic acid can be at least about 100 mg per day, at least about 250 mg per day, at least about 375 mg per day, at least about 500 mg per day, about 750 mg per day, or about 1000 mg per day. Higher daily doses up to about 2000 mg per day can further enhance the effects of the methods. Effective amounts can also be from one to another of any of the preceding values.

In one embodiment, the administering of phosphatidic acid is carried out on a daily basis for at least six weeks or at least eight weeks. Longer substantially continuous administration, e.g. on a daily basis, or at least four days per week, for more than eight weeks, can further enhance the effects of the methods. In other embodiments, the methods are effective when the PA or LPA is administered on days when the subject engages in resistance training or endurance training, such as distance running, cycling, or swimming.

The invention also provides a method of reducing body fat composition in a subject comprising orally administering to a subject an effective amount of phosphatidic acid on a substantially daily basis for at least two weeks, or at least three weeks, or optionally longer periods of time. The administration of the phosphatidic acid can be in conjunction with participation in resistance training, for example, at least three times per week during the phosphatidic acid administration. The decreased body fat in the subject can be accompanied by an increase in skeletal muscle hypertrophy, an increase in muscle hypertrophy of the rectus femoris muscles and pectoral muscles, and/or an increase in lean body mass.

In some embodiments, the administration of phosphatidic acid can be carried out on a daily basis for at least about eight weeks. Additionally, the resistance training during the phosphatidic acid administration can be carried out for at least eight weeks. The decrease in body fat in the subject can be a decrease of at least 0.5% of the subject's body fat, at least 1% of the subject's body fat.

The invention further provides a method for increasing the rate of fat oxidation and/or fatty acid oxidation in a mammal, such as a human. The methods include administering an effective amount of phosphatidic acid to a subject, as described herein. The effects can include improvements in total cholesterol, and improvements in HDL and LDL levels.

The methods relating to the administration of phosphatidic acid can also be carried out in combination with other ingredients. The phosphatidic acid can also be replaced in various embodiments with an mTOR activator to provide the same or similar benefits of the described methods. Examples of such mTOR activators include a composition that includes one or more of lyso-phosphatidic acid, glycerol-3-phosphate, and phospholipase D, optionally in combination with creatine. The phosphatidic acid or phosphatidic acid-enriched phospholipids can be prepared from various plants, seeds and products such as from soybeans, peanuts, wheat, oats, sunflower, safflower, fish, milk, bovine liver, eggs or egg yolks. Creatine can be present in a composition described herein in a ratio of about 5 to about 3 with respect to the phosphatidic acid, phosphatidic acid-enriched phospholipids, or lyso-phosphatidic acid.

The amount of phosphatidic acid, phosphatidic acid-enriched phospholipids, or lyso-phosphatidic acid in a composition can be, for example, about 0.1 grams to about 40 grams, typically about 0.5 to about 2 grams, taken daily. Composition can also include about 50 mg to about 1 gram of phospholipase D (PLD). Creatine can be taken with the PA or LPA, for example, at about 3 grams to about 10 grams, per day. These amounts can also be administered one to about three times per day. The compositions can further include one or more nutritional supplements, and the composition can be in a form for oral administration, such as a tablet or capsule, or a powder that can be added to water or a sports drink.

The invention thus provides methods for increasing muscle mass and strength in mammals comprising orally administering an effective amount of a composition as described herein. The invention also provides methods for improving the nitrogen balance of an aging, bedridden or cachectic human comprising the administration of a therapeutically effective amount of essentially pure phosphatidic acid, phosphatidic acid-enriched phospholipids, lyso-phosphatidic acid, or a combination thereof, or a composition as described herein. The invention further provides methods for increasing the response to the administration of creatine, and methods for reducing body fat.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Lean Body Mass 4 Weeks of LPA Supplementation. For each subject, the left bar is at Week 0 and the right bar is at Week 4.

FIG. 2. Changes in rectus femoris muscle cross sectional area (CSA) between pre- and post-experiment values (cm²).

FIG. 3. Changes in lean body mass (LBA) (kg) between pre- and post-experiment values.

FIG. 4. Changes in body fat mass (kg) between pre- and post-experiment values.

FIG. 5. Analysis of cell plating studies according to Example 7.

FIG. 6. CHEMI Nutra PA activates mTOR signaling. C₂C₁₂ myoblasts were stimulated with various doses of glucose-3-phosphoate (G3P), CHEMI Nutra PA (CN PA) or the vehicle (control) for 20 minutes as described in Example 7. Stimulations with C8 PA or Egg PA were used as positive controls. (A). Western blot of p70 phosphorylated on the threonine 389 residue [P-p70(389)] was compared to total p70 and used as a marker of mTOR signaling. (B). Graphical representation of the P-p70(389) to total p70 ratio expressed as a percent of the control values. * P<0.05 compared to control.

FIG. 7. C₂C₁₂ myoblasts were stimulated with 10 μM or 30 μM doses of phosphatidylserine (PS), phosphatidylinositol (PI), phosphatidylethanolamine (PE), phosphatidylcholine (PC), Chemi Nutra PA (CN PA) or the vehicle (control) for 20 minutes. (A). Western blots of p70 phosphorylated on the threonine 389 residue [P-p70(389)] were compared to total p70 and used as a marker of mTOR signaling. (B). Graphical representation of the P-p70(389) to total p70 ratio expressed relative to the values obtained in the control samples.

FIG. 8. C₂C₁₂ myoblasts were stimulated with 10 μM or 30 μM doses of diacylglycerol (DAG), glycero-3-phosphoate (G3P), lysophosphatidic acid (LPA), egg-PA, Chemi Nutra PA (CN PA) or the vehicle (control) for 20 minutes. (A). Western blots of p70 phosphorylated on the threonine 389 residue [P-p70(389)] were compared to total p70 and used as a marker of mTOR signaling. (B). Graphical representation of the P-p70(389) to total p70 ratio expressed relative to the values obtained in the control samples.

FIG. 9. Simple test gel confirming that egg PA and the Chemi Nutra PA solutions generated had similar concentrations of PA. Based on these results, the Chemi Nutra PA was slightly more concentrated than the Egg PA. However, the minor concentration difference is insufficient explain the robust activation of mTOR by the Chemi PA as shown in FIG. 8.

FIG. 10. MS/MS Parameters.

FIG. 11. Comparison of LPA content (μM) for plasma time points.

FIG. 12. Comparison of PA content (μM) for plasma time points.

FIG. 13. Comparison of summed LPA and P A content (μM) for plasma time points.

### DETAILED DESCRIPTION OF THE INVENTION

Phospholipids occur widely throughout the plant and animal kingdoms. For example, the human spinal cord contains 6-10% and the human brain 4-6% lecithin by weight (w/w). Soybeans are the most important and economical source of commercial lecithin, which has many applications in foods and industrial processes. Although the following examples use lecithin (about 1.5 to about 3.1% w/w) and PA-enriched lecithin from soybeans (10% to 60% w/w), various embodiments of the invention can include lecithin, essentially pure PA, and/or PA-enriched lecithin from any source, including but not limited to peanuts (1.11% w/w), calf liver (0.85% w/w), wheat (0.61% w/w), oatmeal (0.65% w/w), or eggs (0.39% w/w). Among refined substances, especially concentrated sources of lecithin include dehydrated egg yolk (14-20% w/w), natural egg yolk (7-10% w/w), wheat germ (2.82% w/w), soy oil (1.8% w/w) and butterfat (1.4% w/w).

Lecithin has been generally recognized as safe (GRAS) by the US FDA since 1979. Lecithin supplementation has been tested by numerous studies in healthy young athletes with no severe side effects (Jäger et al, 2007 J. Internat. Soc. of Sports Nutrition, 4:5). The effect of lecithin on lowering cholesterol levels (Cobb, 1980 Nutr. Metab. 24:228-237) has been studied. The daily consumption of lecithin in those studies, i.e., 22.5 grams per day for four weeks, contained from 0.4 to 0.7 grams of PA, compared to the 1.6 grams of PA or PA-enriched lecithin per day for four to eight weeks, as is described in Example 1 below. Interestingly, Cobb reports that no PA was found in plasma after 21 days of supplementation, verifying the ephemeral nature of PA as a result of normal metabolism (Cobb, page 232; Table III). At high lecithin levels, undesirable side effects of lecithin may include gastrointestinal distress, nausea and increased salivation.

### The Metabolic Role of Phosphatidic Acid

The biological importance of phosphatidic acid (PA) is becoming increasingly recognized. PA is a common phospholipid in mammals and is a constituent of all cell membranes. Increasing available blood levels of PA can improve membrane stability. However, PA as a cell membrane component is a minor constituent of the total phospholipid pool. PA is the smallest of the phospholipids on a molecular weight basis but is important because it acts as a major precursor to the other phospholipids, many of which are crucial for membrane health. PA is also a key and crucial second messenger in muscular contraction, and muscle cell growth and development. Although PA can be found in the food supply in minor amounts and is a natural component formed during digestion, its existence is ephemeral due to further degradation and entry into the phospholipid synthetic cycle. Before the work described herein, it was unknown whether oral PA could raise systemic PA levels. The inventors have discovered that dietary supplementation with either PA, LPA, or a combination thereof, does in fact raise systemic PA levels and provides significant body composition benefits, as described herein.

In addition to its structural role, PA is an important controller of protein synthesis. The pathways that regulate PA concentration in response to mechanical demand are as yet not fully defined, especially in the intact body. Under normal conditions, the concentration of PA depends on phospholipase D (PLD) enzyme activity, which causes the hydrolysis of phosphatidylcholine, a major membrane component, to PA and choline. PA then binds the FKBP12-rapamycin binding (FRB) domain of the protein mTOR and activates p70S6K, which is one of the key ribosomes of the protein translation phase of protein synthesis. Blocking mTOR with the antibiotic rapamycin has been shown to block protein translation and stop upregulation in response to mechanical stimulation, thereby inhibiting muscle growth.

In vitro studies with skeletal muscle stretch models, cell lysates, or intact cell lines support that PA has an important role in muscle metabolism. Signaling by the mammalian target of rapamycin (mTOR) is reported to be one aspect necessary for mechanical load-induced growth of skeletal muscle, known as muscular hypertrophy. The exact mechanisms for the mechanical activation of mTOR are not known. However, several studies indicate that both phospholipase D (PLD) and PA acting as a second messenger play important roles in the activation of mTOR signaling (Hornberger, et al. Cell Cycle, 5, 1391-1396, 2006; Foster, Cancer Research, 67, 1-4, 2007).

The mechanism of mTOR activation is generally understood to proceed as follows. PA binds to the FKBP12-rapamycin binding (FRB) domain of the protein mTOR and activates p70S6K, a ribosomal dual pathway signaling kinase, which is a key ribosome of the translation phase of protein synthesis. It has been shown that the role PA plays is critical to the synthesis of protein, particularly skeletal muscle proteins. In in vitro studies, an elevation in PA concentration was sufficient for the activation of mTOR signaling. Second, mechanical stimulation results in muscle growth. The mechanical stimulation can include events such as weight lifting-induced PLD activation, PA accumulation, or mTOR signaling. When PLD was blocked, PA did not accumulate and mTOR signaling was prevented.

Interestingly, further studies have indicated that PA binds to and activates p70S6K directly even in the absence of mTOR (Lehman et al. FASEB J. vol. 21, 1075-1087, 2007). This suggests that PA can have an anabolic potential at other times of the day regardless of whether mechanical activation takes place. This finding is of importance in the case of the cachectic, bedridden, or elderly patient who is unable to perform sufficient exercise to induce mechanical activation of mTOR signaling.

Research has also revealed that adenosine monophosphate-activated protein kinase (AMPK) can inhibit mTOR signaling through the phosphorylation of TSC2, an upstream regulator of mTOR (Inoki et al. 2003 Genes Dev. 17: 1829-1834). PA has been shown to increase AMPK activity, which can result in the inhibition of mTOR activity (Kimball 2007 Biochem. Soc. Trans. 35, part 5: 1298-1301). Moreover, AMPK activation has been linked to the reduction of p70S6 kinase activity. Therefore, because AMPK inhibits protein synthesis by a number of different pathways, it is likely that AMPK is a key regulator of cardiac hypertrophy. These results are contrary to the earlier findings and suggest that PA could actually decrease protein synthesis.

Because of the well-known gastrointestinal degradation and entry into the phospholipid synthetic cycle upon uptake into the vascular system, only actual in vivo experimentation can determine if the administration of PA directly leads to increased muscle protein synthesis in mammals, and previous to the inventors' work, oral administration of PA had not been studied. The mechanisms of muscle growth are complex with many factors contributing to the optimal compositions and methods for promoting growth. In addition to signaling at the gene level, good muscle health begins with adequate nutrition. An individual's diet can be optimized to provide an optimum combination of nutrients if the correct combination of nutrients is known. Supplements are available to increase the metabolism of carbohydrates, proteins and fats. For example, whey protein is a complete protein, containing the proper balance of essential amino acids, and is easily digested. Partially hydrolyzed collagen is another complete protein and is even more easily digested. An athlete in a muscle building phase can require 20 to 120 grams of protein daily. Protein supplement of an easily digestible protein such as whey or collagen is even more beneficial for the aging, cachectic or bedridden person.

### Composition Additives and Dietary Combinations

The compositions and methods described herein can benefit from combining phosphatidic acid (PA) with α-lipoic acid. Combinations of PA with certain herbal products such as Russian tarragon, Cissus quadruangularis or Gymnema sylvestre can also be beneficial. Other beneficial products in combination with PA include certain amino acids, creatine, L-carnitine, glycine propionyl-L-carnitine, bitter melon, cissus quadrangularis, cinnamon and fenugreek, creatinol-O-phosphate, leucine, leucine peptides, CLA, tribulus, ribose, caffeine, beta alanine, ZMA, betaine (trimethylglycine (TMG)), L-aspartic acid and carnosine, alone or in combination. Each of these supplements, acting at a different level of metabolism, can enhance the effect of the PA compositions described herein.

Creatine is a leading recommended nutritional supplement, particularly for individuals seeking to maintain or increase muscle mass. Creatine is available in several forms such as creatine salt, creatine ester, creatine ether, creatinol, creatinol ether, creatinol salt, each of which can be advantageously used with the compositions and methods described herein. Various forms of creatine that may be used are described in U.S. Pat. Nos. 7,772,428 (Heuer et al.) and 7,476,749 (Heuer et al.).

Creatine is phosphorylated by creatine kinase (CK) to form an energy reservoir, especially in muscle tissue, for the resynthesis of ATP expended during exercise. Research has shown that the increase in intramuscular creatine levels with creatine supplementation is variable, with mammals falling into “responder” or “nonresponder” groups. Much of this variability can lie within the regulation and activity of the creatine transporter. In one study, approximately 20% to 30% of participants following a creatine loading regimen did not respond with an increase in intracellular creatine (Greenhaff et al. 1994 Amer. J. Physiol. 266 (5Pt 1): E725-30). Another study conducted a descriptive profile of the characteristics of individuals portraying Greenhaff's classification of responders versus nonresponders. Because mTOR has been shown to stimulate the creatine transporter SLC6A8 through mechanisms at least partially shared by the serum and glucocorticoid-induced kinase SGK1, creatine supplementation combined with PA can show a synergistic effect, not only in extending the benefits of creatine supplementation to creatine non-responders, but also to increase the creatine effect in responders. Thus, a composition of creatine with a PA composition can provide significant benefits.

Beyond nutritional supplements, hormones such as testosterone, human growth hormone, insulin, and insulin-like growth hormones can also play a role in promoting anabolism. These hormones may be especially efficacious for cachectic patients. Other “micronutrients” such as chromium, vanadium and coenzyme Q₁₀ (CoQ₁₀) may also be added to the diet or the compositions described herein.

### Phosphatidic Acid Methods

Signaling through mTOR is likely necessary for mechanically induced growth of skeletal muscle and mTOR signaling likely requires a threshold concentration of PA. Until this disclosure, it was unknown whether PA could be sufficiently raised by ingestion by mammals to affect and significantly amplify the growth signaling cascade. Surprisingly, it has been found that PA amplifies the growth signaling cascade, even in the absence of mechanical induction. Thus, PA is important in shifting the metabolism from the catabolic state to the anabolic state and improving nitrogen balance. The shift in metabolism from the catabolic state to the anabolic state can be readily measured by determining the nitrogen balance, the ratio between nitrogen consumed as protein and nitrogen excretion as urea. Alternatively, the urinary excretion of creatinine may be analyzed to determine the nitrogen balance. A positive nitrogen balance is critical for preventing, reducing, or inhibiting cachexia and other forms of muscle deterioration. The oral administration of phosphatidic acid or other actives described herein that provide in situ phosphatidic acid, such as LPA and PS, can increase muscle hypertrophy, strength, and lean body mass, and decrease body fat in subjects. The effects can be achieved in the absence of resistance training. However, resistance training can significantly accelerate and increase the effects.

### DEFINITIONS

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more components in a formulation can refer to one to five, or one to four, or one to three.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for weights of components, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a condition or disorder, or to treat the symptoms of the condition or disorder, in a host. Thus, an “effective amount” generally means an amount that provides the desired effect.

In some embodiments, “an effective amount” can refer to an amount effective for enhancing lean muscle stimulus, growth, strength and recovery, increasing nutrient delivery and/or promoting increased vascular response, e.g., blood flow and circulation, in a subject, for example, when administered over a period of week(s). An effective amount of the actives (e.g., creatine, essentially pure phosphatidic acid, phosphatidic acid-enriched lecithin, lyso-phosphatidic acid, or phospholipase D) can be from about 0.1 g to about 20 g of a nutritional composition per serving. In various embodiments, an effective amount of the composition comprises from about 1 g to about 10 g of the nutritional composition per serving.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The phrase “a substantially daily basis” and the like, refer to administration of an active agent, such as phosphatidic acid, that is on average, representative of a daily frequency. Administration on a substantially daily basis will generally be on a daily basis where an occasional dose may be omitted, for example, one dose once every ten or twelve days. In the context of administration on a substantially daily basis, “substantially continuous administration” refers to the daily frequency of the administration, not that the actual administration is itself continuous, i.e., on a substantially continuous basis at a frequency of about once or twice per day.

The term “resistance training” or “RT” refers to exercise that is performed to provide significant tension on muscle fiber, such as the tension provided by weight-bearing exercises, including but not limited to, weight lifting, weight machine training, and body weight-bearing exercises such as pushups, pullups, and plyometrics. Resistance training, or strength training, is physical activity or exercise that uses the force of a muscle against some form of resistance to build muscle strength, endurance, and/or size. Resistance training can be exercise that is more vigorous than merely walking or other activity that typically does not raise the heart rate to greater than 70% of an individual's maximum heart rate for more than approximately one minute. Resistance training is typically a repetitive exercise focused on exerting a single muscle group or a series of muscle groups, often to exhaustion or at least to the point of feeling the presence of a burning sensation in the muscles being exercised. While resistance training supplements the benefits of PA supplementation, it is not always required; similar body composition benefits can be achieved by larger doses of PA or its related compounds, and/or supplementing the diet with the compounds for a longer period of time, for example, one month, two months, or at least three months.

Lecithin is the commercial term for a naturally occurring mixture of phospholipids (also called phosphatides or phosphoglycerides). The most common phospholipids in lecithin are phosphatidic acid (PA), phosphatidylethanolamine (PE), phosphatidylcholine (PC), phosphatidylserine (PS), and phosphatidylinositol (PI). The “head” of a phospholipid is hydrophilic, while the hydrophobic “tails” are repelled by water and form aggregates in aqueous compositions. As a result of this configuration, phospholipids form natural barriers, segregating or insulating structures. The hydrophilic head contains the negatively charged phosphate group, and may contain other polar groups such as choline. The hydrophobic tail consists of long fatty acid hydrocarbon chains, such as those described below.

Lecithin is found in many natural products including but not limited to soybeans, peanuts, eggs, grains, liver, fish, legumes, safflower, and milk. A typical lecithin used in the compositions and methods described herein is soy lecithin. Lecithin is comprised of phospholipids including phosphatidic acid (PA), however the PA content in lecithin is too low to provide practical amounts of PA for most subjects. Accordingly, this disclosure provides a form of PA that comprises enzymatically processed lecithin resulting in substantially higher wt. % content of PA. The lecithin used to prepare the PA-enriched phospholipid composition can be from any source, including egg or soy. The PA-enriched phospholipid can be provided as a high weight percentage composition, such as at least 40 wt. % PA, at least 45 wt. % PA, at least 50 wt. % PA, or at least 60 wt. % PA. In various embodiments, a suitable composition can be prepared from soy lecithin to contain at least about 40 wt. % PA to provide PA-enriched phospholipid. Minor components of PA-enriched lecithin can be, for example, 5-15% phosphatidylcholine, 1-5% lyso-phosphatidylcholine, 1-5% lyso-phosphatidic acid, and/or 1-5% N-acyl phosphatidylethanolamine. These components do nor interfere with the PA activity and in some cases may augment the PA activity. The source lecithin can include chemically or enzymatically altered derivatives, such as DHA-soy lecithin, thereby providing a PA-enriched phospholipid composition with high amounts of DHA (e.g., at least 10 wt. %, at least 20 wt. %, or at least about 30 wt. %).

The term “enriched” refers to a partially purified extract or composition from which undesirable impurities have been removed and/or by certain actions, the concentration of a particular component is increase. The undesirable impurities may be a single compound or multiple compounds. In some embodiments, at least 25%, at least 50%, or at least 75%, of the undesirable impurities have been removed.

Phosphatidic acid (PA) is a diacyl-glycerophospholipid and a major constituent of cell membranes. The structure of PA is:

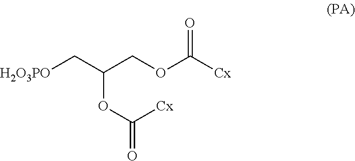

where Cx is the alkyl or alkenyl moiety of a fatty acid. Typically, an unsaturated fatty acid bonded to carbon-2 and a saturated fatty acid is bonded to carbon-3. PA can also be used in the compositions described herein as its phosphate salt.

Essentially pure PA refers to a composition of phosphatidic acid (PA) that is at least about 95%, at least about 96%, at least about 97%, at least about 98%, at least about 99%, or at least about 99.5%, pure PA, by weight. PA-enriched phospholipid includes at least about 10%, at least 40%, at least 50%, at least 60%, or at least 70%, PA.

Lysophosphatidic acid (lyso-PA or LPA) is a phospholipid having the structure:

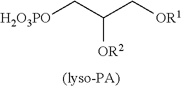

where one R¹ is the alkanoyl or alkenoyl moiety of a fatty acid and R² is H, or vice versa. When R¹ is H, the compound is 1-lyso-PA; when R² is H, the compound is 2-lyso-PA. Examples of lyso-PA fatty acid moieties (e.g., OR¹ or OR²) include (Z)-octadec-9-enoate or octadecanoate (stearate). Examples of fatty acids that can form esters with the glycerol backbone of PA or lyso-PA include, but are not limited to, decanoic acid (10:0), undecanoic acid (11:0), 10-undecanoic acid (11:1), lauric acid (12:0), cis-5-dodecanoic acid (12:1), tridecanoic acid (13:0), myristic acid (14:0), myristoleic acid (cis-9-tetradecenoic acid, 14:1), pentadecanoic acid (15:0), palmitic acid (16:0), palmitoleic acid (cis-9-hexadecenoic acid, 16:1), heptadecanoic acid (17:1), stearic acid (18:0), elaidic acid (trans-9-octadecenoic acid, 18:1), oleic acid (cis-9-octadecanoic acid, 18:1), nonadecanoic acid (19:0), eicosanoic acid (20:0), cis-11-eicosenoic acid (20:1), 11,14-eicosadienoic acid (20:2), heneicosanoic acid (21:0), docosanoic acid (22:0), erucic acid (cis-13-docosenoic acid, 22:1), tricosanoic acid (23:0), tetracosanoic acid (24:0), nervonic acid (24:1), pentacosanoic acid (25:0), hexacosanoic acid (26:0), heptacosanoic acid (27:0), octacosanoic acid (28:0), nonacosanoic acid (29:0), triacosanoic acid (30:0), trans vaccenic acid (trans-1′-octadecenoic acid, 18:1), tariric acid (octadec-6-ynoic acid, 18:1), and ricinoleic acid (12-hydroxyoctadec-cis-9-enoic acid, 18:1) and ω3, ω6, and ω9 fatty acyl residues such as 9,12,15-octadecatrienoic acid (α-linolenic acid) [18:3, ω3]; 6,9,12,15-octadecatetraenoic acid (stearidonic acid) [18:4, ω3]; 11,14,17-eicosatrienoic acid (dihomo-.alpha.-linolenic acid) [20:3, ω3]; 8,11,14,17-eicosatetraenoic acid [20:4, ω3], 5,8,11,14,17-eicosapentaenoic acid [20:5, ω3]; 7,10,13,16,19-docosapentaenoic acid [22:5, ω3]; 4,7,10,13,16,19-docosahexaenoic acid [22:6, ω3]; 9,12-octadecadienoic acid (linoleic acid) [18:2, ω6]; 6,9,12-octadecatrienoic acid (γ-linolenic acid) [18:3, ω6]; 8,11,14-eicosatrienoic acid (dihomo-γ-linolenic acid) [20:3 ω6]; 5,8,11,14-eicosatetraenoic acid (arachidonic acid) [20:4, ω6]; 7,10,13,16-docosatetraenoic acid [22:4, ω6]; 4,7,10,13,16-docosapentaenoic acid [22:5, ω6]; 6,9-octadecadienoic acid [18:2, ω9]; 8,11-eicosadienoic acid [20:2, ω9]; 5,8,11-eicosatrienoic acid (Mead acid) [20:3, ω9]; trans-10,cis-12 octadecadienoic acid; cis-10,trans-12 octadecadienoic acid; cis-9,trans-11 octadecadienoic acid; trans-9,cis-11 octadecadienoic acid, as well as “omega-3 fatty acids” such as Δ-5,8,11,14,17-eicosapentaenoic acid (EPA), Δ-4,7,10,13,16,19-docosahexanoic acid (DHA) and Δ-7,10,13,16,19-docosapentanoic acid (n-3 DPA). The acyl residues of a fatty acid moiety can also be conjugated alkenes, hydroxylated, epoxidized, and/or hydroxyepoxidized acyl residues.

Lyso-PA can act as a signaling molecule. Lyso-PA can be used in the compositions described herein as its phosphate salt. Lyso-PA can be substituted for PA in any embodiment of this invention, or it can be added to a composition or method that uses PA to supplement the composition's activity.

Mediator® phospholipids is a high phosphatidic acid containing mixture of phospholipids prepared by enzymatic conversion of lecithin to primarily phosphatidic acid. The composition can include at least about 50 wt. % PA and at least about 5 wt. % LPA. In some embodiments, the composition can include about 60-70 wt. % PA, about 6-10 wt. % LPA, about 4-10 wt. % phosphatidylcholine, about 4-10 wt. % phosphatidylethanolamine, about 2-8 wt. % phosphatidylinositol, and about 1-5 wt. % lyso-phosphatidylcholine. The composition can be provided in capsules or the like, containing about 400 mg of phospholipids with approximately 200 mg of PA (typical minimum value), optionally in combination with excipients or carriers such as rice flour and/or magnesium stearate. The various phospholipid compositions described herein can exclude any number of components that are not themselves phospholipids. Thus, the phospholipids can be in the form of a discrete dosage unit that consists essentially of phospholipids and optionally inert diluents, carriers, or excipients.

Phospholipase D (PLD) is an enzyme that catalyzes the hydrolysis of phosphatidylcholine to form phosphatidic acid (PA), releasing the soluble choline headgroup into the cytosol. PLD is often located in the plasma membrane of cells. The two mammalian isoforms of phospholipase D are PLD1 and PLD2. One specific example of PLD is autotaxin, also known as ectonucleotide pyrophosphatase/phosphodiesterase family member 2 (E-NPP 2). Autotaxin has lysophospholipase D activity that converts lysophosphatidylcholine into lyso-PA. PLD can be substituted for PA in any embodiment of this invention, or it can be added to a composition or method that uses PA to supplement the composition's activity. The amount of PLD used can be about 50 mg to about 1 gram, about 100 mg to about 800 mg, about 200 mg to about 750 mg, about 100 mg to about 400 mg, about 400 mg to about 800 mg, or 200 mg, 400 mg, 500 mg, 750 mg, or 1 gram.

Creatine refers to the chemical compound N-methyl-N-guanyl glycine (CAS Registry No. 57-00-1), also known as (α-methyl guanido)acetic acid, N-(aminoiminomethyl)-N-glycine, methylglycocyamine, methylguanidoacetic acid, and N-methyl-N-guanylglycine, whose chemical structure and zwitterionic form are shown below.

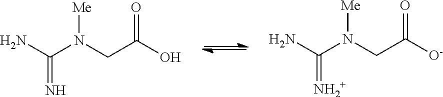 [SMILES: CN(CC(=O)O)C(=N)N ; CN(CC(=O)[O-])C(N)=[NH2+]]

As used herein, “creatine” also includes derivatives of creatine such as esters, and amides, and salts, as well as other derivatives, including derivatives that become active upon metabolism. to Creatinol (CAS Registry No. 6903-79-3), also known as creatine-O-phosphate, N-methyl-N-(beta-hydroxyethyl)guanidine O-phosphate, Aplodan, or 2-(carbamimidoyl-methyl-amino)ethoxyphosphonic acid, is also a creatine derivative that can be used in the compositions and methods described herein.

Creatine and creatine derivatives are widely available from a number of commercial sources. Commercially available creatine derivatives include creatine phosphate, creatine citrate, magnesium creatine, alkaline creatine, creatine pyruvate, creatine hydrates (including, but not limited to creatine monohydrate), and creatine malate. Glycocyamine, an in vivo precursor of creatine, is also commercially available and suitable in the practice of the invention described herein. In some embodiments, the compositions include creatine malate or creatine monohydrate.

The “anabolic window” or the “metabolic window” refers to the first 90 minutes following vigorous exercise, when the body is typically in a catabolic state as a result of the exercise. During this window the metabolism of protein can be extremely rapid. Catabolism of protein can be even more enhanced in the first 45 or first 90 minutes following vigorous exercise. However, proper consumption of protein and other nutrients can shift the body from a catabolic state to an anabolic state during the anabolic window, thereby enhancing muscle building, strength gains, and muscle recovery.

### Compositions and Methods of the Invention

The invention provides novel compositions comprising high amounts of phosphatidic acid (PA). The invention also provides methods for using the compositions, such as for increasing strength and lean muscle mass. Compositions having a therapeutically effective amount of PA or PA-enriched phospholipid sufficient to affect intracellular and extracellular concentrations of PA in a mammal can shift the metabolism from a catabolic state to an anabolic state. This shift counteracts the decrease in muscle tissue that occurs with normal aging and other conditions such as bed rest, cachexia, and weightlessness. The compositions and methods can also increase exercise capacity in normal healthy mammals where increased muscle mass and strength is often desired.

The administration of PA in combination with resistance training, such as a weight training program or other anaerobic exercise routines, can further potentiate the effects of PA administration. The effects of the PA administration can be increased by administering the PA to an exercising individual within the anabolic window, when the effect of ingesting PA is more pronounced. These methods also can benefit from protein ingestion at approximately 90 minutes before exercise or less, and within about 45 or 90 minutes post-exercise. An easily digested protein supplement such as whey protein can increase the effect. Suitable amounts of protein can be, for example, about 10 grams to about 50 grams, per hour of intense exercise, in one or more doses. The protein can be for example, whey protein or partially hydrolyzed collagen.

The combination of creatine and PA can also be used in a composition to increase muscle mass. Creatine is stored mainly in muscle tissue, where it is phosphorylated to creatine phosphate by ATP. The high energy phosphate bond of creatine phosphate is readily transferred to adenosine diphosphate by the enzyme creatine kinase forming ATP, which is available for muscle contraction and relaxation. Thus creatine phosphate may be considered a reservoir of muscle energy. Creatine is readily available as a nutritional supplement. However, about 30% of humans are creatine non-responders. In these subjects, no creatine is found in the tissues after creatine supplementation. PA has been found to switch creatine non-responders to creatine responders by a yet unknown mechanism. The dosage of creatine sufficient to switch non-responders to be able to respond to creatine supplementation is generally about 3 grams to about 20 grams per day for at least about five days.

The invention further provides a composition that includes one or more of essentially pure phosphatidic acid, phosphatidic acid-enriched phospholipid, lyso-phosphatidic acid, glycerol-3-phosphate, phospholipase D, phosphatidylserine, or diacylglycerol (DAG), optionally in combination with creatine. The essentially pure phosphatidic acid or phosphatidic acid-enriched phospholipid can be prepared from soybeans, peanuts, wheat, oats, safflower, fish, milk, bovine liver, eggs or egg yolks. The composition can include, alone or in addition to phosphatidic acid, phosphatidylserine, phosphatidylethanolamine, phosphatidylinositol, phosphatidylcholine, and/or lyso-PA. These components can be, for example, soy-derived or egg-derived. When included in the composition, the creatine can be present in a ratio of about 2:1 or about 1.4:1 with respect to the phosphatidic acid, phosphatidic acid-enriched phospholipid, lyso-phosphatidic acid, or the combination thereof.

The composition can include about 50 mg to about 1 gram of phospholipase D (PLD). The amount of essentially pure phosphatidic acid, phosphatidic acid-enriched phospholipid, or lyso-phosphatidic acid can be 0.1 grams to about 40 grams. The amount of creatine can be, for example, about 3 grams to about 10 grams. Each of these amounts can be administered one to about three times per day.

The compositions can further include one or more nutritional supplements. Examples of such supplements include, but are not limited to, protein, one or more amino acids such as leucine or L-aspartic acid, beta alanine, leucine peptide, α-lipoic acid, β-hydroxy-β-methyl butyrate (HMB), carnitine, glycine propyline-L-carnitine, Russian tarragon, gymnema sylvestre, bitter melon, cissus quadrangularis, cinnamon and fenugreek, CLA, tribulus, mulberry, ribose, caffeine, ZMA, betaine, carnosine, or a combination thereof. Additional additives can include Co-Q10, chromium, magnesium, vanadium, or a combination thereof. The composition can be in a form for oral administration, such as a tablet, capsule, or powder.

The invention also provides methods for increasing muscle mass and strength in mammals comprising orally administering an effective amount of a composition as described herein. The composition can include, for example, an effective amount of essentially pure phosphatidic acid, phosphatidic acid-enriched phospholipid, lyso-phosphatidic acid, or a combination thereof. The effective amount can be 0.1 grams to about 40 grams, administered orally one to three times daily. For example, in a human, the effective amount can be 0.5 to about 5 grams. In a canine, the effective amount can be 0.1 to about 3 grams, and in an equine, the effective amount can be about 10 to about 40 grams.

The active or combination of actives (e.g., PA, lyso-PA, PLD, G3P, or a combination thereof) can be administered to an exercising or previously exercised subject during the anabolic window. The method can also include the ingestion of 20 to 100 grams of protein during the anabolic window. The protein can be, for example, a complete protein containing all the essential amino acids for humans, horses, or dogs. In one embodiment, the protein is whey or partially hydrolyzed collagen protein.

The invention also provides methods for improving the nitrogen balance of an aging, bedridden or cachectic human comprising the administration of a therapeutically effective amount of essentially pure phosphatidic acid, phosphatidic acid-enriched phospholipid, lyso-phosphatidic acid, or a combination thereof, or a composition as described herein. The effective amount can be 0.1 grams to 4 grams, given orally one to four times daily. The effective amount can be administered orally or by parenteral infusion. The method can further include the administration of an effective amount of creatine, such as about 3 to about 20 grams.

The invention further provides methods for increasing the response to the administration of creatine. The methods can include administering an effective amount of essentially pure phosphatidic acid, phosphatidic acid-enriched phospholipid, lyso-phosphatidic acid, or a combination thereof, concomitant with the co-administration of an effective amount of creatine, to a human subject 1 to 3 times daily. The effective amount of can be 0.5 to about 4 grams, and the effective amount of creatine can be about 3 to about 10 grams.

The invention yet further provides methods for increasing muscle mass and strength in mammals comprising the administration of an effective amount of a composition as described herein, as well as methods for increasing muscle mass and strength in mammals comprising administering an effective amount of a composition as described herein in combination with a hormone. The hormone can be, for example, testosterone, human growth hormone, insulin, or insulin-like growth factor.

Experiments described in the examples below show that in addition to phosphatidic acid, phosphatidylserine also activates mTOR. Accordingly, phosphatidylserine can be used in methods of the invention to achieve results similar to phosphatidic acid. Furthermore, while some mTOR activators are not as active as PA or LPA, certain combinations can be used to provide similar effects, such as the combination of G3P and lecithin or lower amounts of PA and/or LPA, or PLD in combination with lecithin or lower amounts of PA and/or LPA (e.g., amounts lower than 500 mg per day or lower than 100 mg per day).

### Formulation Components

The compounds and active agents described herein can be used to prepare therapeutic compositions. The compounds may be added to the compositions in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The actives described herein can be formulated as therapeutic compositions and administered to a mammalian host, such as a human patient, in a variety of forms. The forms can be specifically adapted to a chosen route of administration, e.g., oral or or subcutaneous routes. The actives may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 1% of active compound. In some embodiments, the percentage of the compositions and preparations can vary and may conveniently be from about 2% to about 90% of the weight of a given unit dosage form. The amount of active in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active may be incorporated into sustained-release preparations and devices.

The active may also be administered as a solution or dispersion. Solutions of the active compound or its salts can be prepared in water, optionally mixed with a nontoxic surfactant. Dispersions can be prepared in pharmaceutically acceptable oils such as glycerol, liquid polyethylene glycols, triacetin, or mixtures thereof. Under ordinary conditions of storage and use, preparations may optionally contain a preservative to prevent the growth of microorganisms.

### Dosage

Useful dosages of the actives described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of an active, an active salt or derivative thereof, or a combination of actives, can vary not only with the particular compound or salt selected but also with the route of administration, the nature of the condition being treated, and the age and condition of the patient, and can be ultimately at the discretion of an attendant physician, clinician, nutritional advisor.

The dose may conveniently be presented in a single dose or as divided doses administered at appropriate intervals, for example, as two, three, four or more doses or sub-doses per day. The sub-dose itself may be further divided, e.g., into a number of discrete loosely spaced administrations.

A dosage of 0.1 grams to about 40 grams of PA-enriched phospholipid can be administered to a mammal orally one to three times daily, preferably 90 minutes before to 90 minutes after exercise, e.g., during the anabolic window. When the mammal is a human, 0.5 to four grams is a recommended single dosage. When the mammal is a horse, 10 to 40 grams is a recommended dosage. When the mammal is a whippet, 0.1 to 0.3 grams is a recommended dosage. When the mammal is a greyhound, 0.2 to 0.4 grams is a recommended dosage.

A gastric acid secretion inhibitory coating may be applied to the dose in a manner that protects the PA from degradation by gastric juices. Examples of such enteric coatings include polymers such as cellulose. Enteric coated PA can be incorporated in the manufacture of foods, drugs, and dietary supplements of complex formulations and various dosage forms including capsules, tablets, caplets, lozenges, liquids, solid foods, powders and other dosage forms that may be developed, without the need to impart enteric protection to the entire mixture, any other part of the mixture, or finished products.

A variety of methods for delivery and/or administration of PA can be carried out, for example and not by way of limitation, by tablet, capsule, powder, granule, microgranule, pellet, soft gel, controlled release form, liquid, solution, elixir, syrup, suspension, emulsion, magma, gel, cream ointment, lotion, transdermal, sublingual, ophthalmic, nasal, aerosol, inhalation, spray, parenteral, suppository and the like. In suitable cases, PA may be administered by intravenous or intraarterial infusion. Compositions of the invention may also be administered in nutraceutical or functional foods. In addition, the effective amount of PA may be combined with amino acids, botanicals, functional foods, herbals, nucleotides, nutraceuticals, pharmaceuticals, proteins, minerals, and/or vitamins in an effort to enhance the targeted activity. Vitamins, amino acids, and other additives that can be combined with PA in various compositions are described by, for example, Remington: The Science and Practice of Pharmacy, 22^{th} edition (Lippincott Williams & Wilkins, 2000).

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention. Thus, the following experiments were carried out to show more clearly the effect of PA administration in increasing muscle hypertrophy and strength. These examples are given in detail in order to more clearly explain how to make and use the invention and do not limit the scope of the appended claims. Human subjects were used, but the results are readily obtained with other mammals such as the horse and the dog, with adjustments in dosage appropriate to body weight.

### EXAMPLES

### Example 1

### Effects of PA Supplementation on Muscle Mass and Training Volume

Enrollment Criteria. A double-blinded study was planned to test the effect of PA on muscle strength. The inclusion criteria were: participation in a resistance training program on a regular basis at recreational level or higher; no physical limitations as determined by health and activity questionnaire; between the ages of 18 and 29. Subjects were excluded if they had allergy to soy, dairy, egg and wheat ingredients, peanuts, seeds and tree nuts. Those taking any other nutritional supplement or performance enhancing drugs were excluded. Finally, subjects were excluded if it was determined they were unable or unwilling to perform the physical exercise to be performed for the study.

Recommended supplements. Essentially pure PA and PA-enriched phospholipid were prepared from soy lecithin by enzymatic conversion. The PA-enriched phospholipid product produced by Chemi Nutra, Inc. (White Bear Lake, Minn.) contains 50-60% phosphatidic acid, 5-15% phosphatidylcholine, 1-5% lyso-phosphatidylcholine and 1-5% N-acyl phosphatidylethanolamine. The PA-enriched phospholipid was given in four 400 milligram capsules to provide 1.6 grams of PA-enriched phospholipid. The placebo was rice flour in a capsule identical in weight and color to the PA-phospholipid capsule.

The methods as described below included a protein snack. Any easily digested protein may be given. The protein used was partially hydrolyzed and termed “collagen protein” having the components described in Table 1. Proline and hydroxyproline comprised about one quarter of the amino acids and the leucine content was low. Collagen protein with low leucine content was chosen because leucine can have an effect on muscle and for clarity, that effect was minimized by choice of protein.

TABLE 1
Collagen Protein Content.

| | g AA/ |
|---|---|
| Amino Acid (AA) | 100 g product |
| Alanine | 7.6 |
| Arginine | 7.8 |
| Aspartic Acid | 5.1 |
| Cysteine | 0.0 |
| Glutamic Acid | 10.5 |
| Glycine | 18.2 |
| Histidine | 1.2 |
| Hydroxylysine | 0.5 |
| Hydroxyproline | 10.8 |
| Isoleucine | 1.4 |
| Leucine | 2.8 |
| Lysine | 3.1 |
| Methionine | 0.6 |
| Phenylalanine | 1.9 |
| Proline | 12.2 |
| Serine | 2.8 |
| Threonine | 1.7 |
| Tryptophan | 0.0 |
| Tyrosine | 0.6 |
| Valine | 2.0 |

Resistance training schedule. A. Four recreationally trained, young, healthy men, with at least one year of resistance training experience who met the enrolment criteria, were recruited for this study. All subjects performed the same training program, four days each week, split routine program as described below in Table 2. The four-day a week workout that was recommended to each subject included core exercises (denoted with an asterisk) which were a requirement of the study. Other exercises, assistance exercises, could be substituted for the core exercises only with the investigator's approval. However, all sets and repetitions were required to be the same in number. Subjects were allowed a 90 second rest period between each set. No additional sets or exercises were allowed as this would change the training volume, defined as the total work load (reps times weight.).

TABLE 2
Eight Week Resistance Training Program.

| | |
|---|---|
| Exercise | Sets/Reps (RM) |

Monday/Thursday

| | |
|---|---|
| Bench Press* | 4 × 10 to 12 |
| Incline Dumbbell Press | 3 × 10 to 12 |
| Seated Shoulder Press* | 4 × 10 to 12 |
| Upright Rows | 3 × 10 to 12 |
| Lateral Raises | 3 × 10 to 12 |
| Shrugs | 3 × 10 to 12 |
| Triceps Pushdown | 3 × 10 to 12 |
| Triceps Extension | 3 × 10 to 12 |
| Situps | 3 × 25 |

Tuesday/Friday

| | |
|---|---|
| Squats* | 4 × 10 to 12 |
| Lunge/Front Squat | 3 × 10 to 12 |
| Leg Curl | 3 × 10 to 12 |
| Knee Extension | 3 × 10 to 12 |
| Calf Raises | 3 × 10 to 12 |
| Lat Pulldown | 4 × 10 to 12 |
| Seated Row | 4 × 10 to 12 |
| EZ Bar Curl | 3 × 10 to 12 |
| Dumbbell Curls | 3 × 10 to 12 |
| Situps | 3 × 25 |

*denotes required exercise

The subjects were randomly divided into two groups, the test group and the control group. The test group received 4 capsules of 400 mg equaling 1.6 grams per day of PA-enriched phospholipid (Mediator®, Chemi Nutra, Inc., White Bear Lake, Minn.). The control subjects received 4 capsules of 400 mg equaling 1.6 grams per day of rice flour. Subjects consumed either the test supplement or the placebo 15 minutes prior to workout. At the end of each workout, subjects were provided with a collagen protein drink consisting of 36 grams of collagen peptides mixed with 500 mL of water. On days of no workout, subjects consumed the respective capsules at approximately the same time of day that they worked out. During these non-workout days, subjects did not receive the protein drink.

At weeks 1 and 7 (pre- and post-study) subjects performed a 1-repetition maximum (1RM) strength test on the squat and bench press exercises. Each subject performed a warm-up set using a resistance that is approximately 40-60% of his perceived maximum and then performed three to four subsequent attempts to determine the 1RM. Subjects were allowed 3 to 5 minutes of rest between each lift. Results are summarized in Table 3.

TABLE 3
Strength Change Data.
Strength (1RM) Bench Press

| PA: average increase: | | | | Placebo: | | | |
|---|---|---|---|---|---|---|---|
| +20 kg (plus 10.5%) | | | | unchanged (0%) | | | |
| | Pre | Post | Delta | | Pre | Post | Delta |
| Subject | 225 kg | 250 kg | +25 kg | Subject | 220 kg | 220 kg | 0 |
| 1 | | | | 3 | | | |
| subject | 155 kg | 170 kg | +15 kg | Subject | 175 kg | 175 kg | 0 |
| 2 | | | | 4 | | | |

Strength (1RM) Squat

| PA: Average increase: | | | | Placebo: | | | |
|---|---|---|---|---|---|---|---|
| +40 kg (+21%) | | | | unchanged +2.5 kg (+1%) | | | |
| | Pre | Post | Delta | | Pre | Post | Delta |
| Subject | 225 kg | 285 kg | +60 kg | Subject | 290 kg | 290 kg | 0 |
| 1 | | | | 3 | | | |
| Subject | 155 kg | 175 kg | +20 kg | Subject | 215 kg | 220 kg | 0 |
| 2 | | | | 4 | | | |

PA supplementation resulted in an increase in strength in the bench press of 10.5% and an increase in strength in the squat of 21%. Supplementation with the placebo resulted in no increase in strength in either exercise.

B. The effects of PA supplementation on muscle mass and training volume were determined in two recreationally trained, young, healthy men, with at least one year of resistance training experience. As above, these subjects received either 1.6 grams per day of Mediator® PA-enriched phospholipid (Chemi Nutra, White Bear Lake, Minn.) or 1.6 grams of rice flour for 6 weeks. Changes in muscle mass were measured by analyzing the muscle thickness of the vastus lateralis, the large lateral muscle on the thigh, using a GE Logiq PS Premium BT09 (Wauwatosa, Wiss). Training volume was calculated as weight lifted times repetitions performed.

The two subjects performed the same 6-week training program, consisting of a 2 day per week lower body resistance program (squats, lunge/front squat, leg curl, knee extension, calf raises, seated row, EZ bar curls, dumbbell curls.) There was a 90 second rest period between each set. The addition of any additional sets or exercises was prohibited as it would change the training volume.

PA supplementation resulted in an increase of 13% in training volume (pre: 49,640; post: 56,000), whereas placebo had no effect on training volume (pre: 92,800; post: 92,800). PA supplementation also resulted in a greater increase in muscle thickness compared to the placebo. In summary, PA supplementation resulted in greater increase in muscle mass, as demonstrated in this study, resulting in an increase of 9% more between a PA supplemented subject and a non-supplemented subject. In addition, PA supplementation resulted in greater increase in training volumes (13%).

Creatine responders.

As discussed above, creatine is a known muscle building substance, but about 30% of any population does not respond to creatine administration.

Subject A.

RJ, a 42-year old male, 198 cm tall, a known non-responder to creatine supplementation, followed a two-week strength training program while testing whether supplementation with PA-enriched-phospholipid improves creatine response. Total starting body weight and strength were measured on day one, followed by the training program, consisting of concentric and eccentric isotonic lifting exercises that worked the upper and lower body muscle groups. Either free weights or weight machines were used once or twice weekly. Strength training was performed three times per week with at least one day of rest between sessions, which alternated between lower and upper body exercises. During the two-week period, a total of six training sessions (three upper and three lower) were performed. Each exercise included two sets of ten repetitions at 30% and 60% 1RM, followed by two sets of 3 to 5 repetitions at 90% 1RM.

The lower body exercise included seven different exercises: seated leg press, leg curls, standing calf raises, leg extensions, inclined leg lift, inverted situps (back extension) and 45° inclined situps. The upper body exercise consisted of seven different exercises: bench press, latissimus pulldown, triceps pulldown, inclined dumbbell curls, seated preacher curls, seated rows, and CyBec Pec Fly.

Total upper body weight and lower body weight lifted were calculated as the average weight lifted during the last three sets multiplied by the average repetition in each set. Total strength was determined as the combined lower and upper total weight lifted. Total body weight was determined after day 8 and day 15. Strength was measured on days 1 and 15.

A four-week rest period followed the first two-week training program. The same program was repeated twice, the control was supplementation of creatine monohydrate (Creapure, Alzchem, Germany) 4 times 5 grams per day for five days of loading, followed by nine days of five grams creatine monohydrate. During the second program, creatine monohydrate was given as for the control program with the addition of PA-enriched phospholipid (Chemi Nutra, White Bear Lake, Minn.) which was about 50% PA. The results are shown in Table 4.

TABLE 4
Changes in Body Mass.

| | No | Creatine | Creatine plus PA-enriched |
|---|---|---|---|
| Day | Supplement | Supplement | phospholipid |
| 1: weight, kg | 90.0 | 88.8 | 88.5 |
| 8: weight, kg | 88.6 | 88.3 | 89.7 |
| 15: weight, kg | 88.2 | 88.0 | 90.1 |

As noted in Table 4, creatine loading alone was not effective in preventing a slight weight loss, while creatine plus PA-enriched phospholipid reversed the weight loss and allowed a slight weight gain, presumably due to an increased muscular creatine concentration with concomitant muscle weight gain, as expected from the known non-responder status of RJ. Looking at total strength substantiated this theory: during the two-week baseline period, strength increased 6%, the same as during the creatine supplement period, which showed a similar, 5% strength increase, verifying that RJ was a creatine non-responder. The supplementation with both creatine and PA-enriched phospholipid showed a gain in strength of 13.4%, more than double that of exercise alone or supplementation with creatine plus exercise.

Subject B.

MP, a 43-year old male, 185 cm tall, also a known non-responder to creatine supplementation, followed a three-week strength training program while testing whether supplementation with PA-enriched-phospholipid improves creatine response. Total starting total body weight and strength were measured on day one, followed by the training program, consisting of concentric and eccentric isotonic lifting exercises that worked the upper and lower body muscle groups with either free weights or weight machines used once or twice weekly. Strength training was performed three times per week with at least one day of rest between sessions, which alternated between lower and upper body exercises. During the three-week period, a total of 10 training sessions (five upper body and five lower body) were performed. Each exercise included two sets of ten repetitions at 40% and 65% 1RM, followed by two sets of 3 to 5 repetitions at 90% 1RM.

The lower body exercise included seven different exercises: seated leg press, leg curls, standing calf raises, leg extension, inclined leg lift, inverted situps (back extension) and 45° inclined situps. The upper body exercise consisted of seven different exercises: bench press, latissimus pulldown, triceps pulldown, inclined dumbbell curls, seated preacher curls, seated rows, and CyBec Pec Fly. Total upper body and lower body weight lifted were calculated as the average weight lifted during the last three sets multiplied by the average repetition in each set. Total strength was determined as the combined lower and upper total weight lifted. Total body weight was determined after day 8 and day 22. Strength was measured on days 1 and 22.

A four-week rest period followed the first three-week training program. The same program was repeated twice, the control was supplemented with creatine monohydrate (Creapure®, Alzchem, Germany), 5 grams per day of five grams creatine monohydrate for 21 days. During the second program, creatine monohydrate was given as for the control program with the addition of 1 gram per day PA-enriched phospholipid (Chemi Nutra, White Bear Lake, Minn.), which contained about 50% PA. The results are shown in Table 5.

TABLE 5
Changes in Body Mass.

| | No | Creatine | Creatine and PA-enriched |
|---|---|---|---|
| Day | Supplement | Supplement | phospholipid |
| 1: weight, kg | 82.0 | 81.9 | 81.5 |
| 10: weight, kg | 81.8 | 81.5 | 82.9 |
| 22: weight, kg | 81.7 | 81.2 | 83.1 |

As noted in Table 5, creatine loading alone was not effective in preventing a slight weight loss, while creatine plus PA-enriched phospholipid reversed the weight loss and allowed a slight weight gain, presumably due to an increased muscular creatine concentration with concomitant muscle weight gain, as expected from the known non-responder status of MP. Looking at total strength substantiated this theory: during the two-week baseline period, strength increased 5%, the same as during the creatine supplement period, which showed a similar 5% strength increase, verifying that MP was a creatine non-responder. The supplementation with both creatine and PA-enriched phospholipid showed a gain in strength of 11.5%, more than double that of exercise alone or supplementation with creatine plus exercise.

### Example 2

### PA-Enriched Phospholipid for the Improvement of Nitrogen Balance

Preliminary studies show that those unable to exercise, such as the bedridden or patients with diseases causing cachexia, can improve their condition with a shift of metabolism from catabolic to anabolic by taking a phosphatidic acid supplement. The primary aspect of cachexia is the loss of protein as a result of muscle breakdown. Because about 60% to 70% of bodily protein is found in muscle and the nitrogen is excreted as urea, measurement of 24-hour urea outcome versus protein nitrogen intake provides the nitrogen balance. When the excretion of nitrogen is greater than the ingestion of protein nitrogen, the patient is said to be in negative nitrogen balance, which can lead to sarcopenia, also known as muscle disuse atrophy.

There are several ways of determining nitrogen balance. First, a diary of foods eaten can be kept and protein intake recorded and compared with a 24 hour collection of urinary nitrogen. This direct measurement is often standard care in hospitals and nursing homes for these patients. Another indicium is 24 hour urinary creatinine. Creatinine is the metabolite of creatine, as noted above, an important compound in muscle. Creatinine is excreted without reabsorption from the kidney tubules and can be determined as an estimate of renal function. Creatinine recovery varies greatly from patient to patient and is affected by such things as degree of hydration. However, once a baseline is established, variations from the patient's idiosyncratic “normal” creatinine excretion are indicative of muscle breakdown. It is also a secondary indicium of nitrogen balance.

The patients will be given 0.5 to four grams of PA-enriched phospholipid three times a day. While oral administration is preferred, for those patients unable to ingest or who are on intravenous or intraarterial therapies, PA or PA-enriched phospholipid may be infused. The results will show an improvement in nitrogen balance.

Supplementation with PA can also increase muscle mass and strength in elderly subjects. Even healthy older subjects may lose muscle to the point of sarcopenia, and some loss of muscle mass may be considered inevitable and irreversible. However, it has been shown that 70-year old adults show a response to the known muscle stimulant β-hydroxy-β-methyl butyrate similar to that of young adults (Vukovich, et al. 2001 Am. Soc. Nutr. Sci. 2049-2053). The compositions and methods described herein can also improve the muscle mass and strength of older subjects. The effects of PA supplementation can be augmented by additionally ingesting of about 5 grams of creatine and 3 grams of an amino acid such as leucine or glutamine, taken 1 to 3 times daily in their exercise regimen, in conjunction with the PA supplementation.

### Example 3

### Phosphatidic Acid Increases Lean Body Mass and Strength in Resistance Trained Men

Phosphatidic acid (PA) is a natural phospholipid compound derived from lecithin, which is commonly found in egg yolk, grains, fish, soybeans, peanuts and yeast. PA is involved in several intracellular processes associated with muscle hypertrophy. Specifically, PA has been reported to activate protein synthesis through the mammalian target of rapamycin (mTOR) signaling pathway and thereby can enhance the anabolic effects of resistance training. To our knowledge, no one has examined the effect of PA supplementation in humans while undergoing a progressive resistance training program.

This example examined the effect of PA supplementation on lean soft tissue mass (LM) and strength after 8 weeks of resistance training.

Fourteen resistance-trained men (mean±SD; age 22.7±3.3 years; height: 1.78±0.10 m; weight: 89.3±16.3 kg) volunteered to participate in this randomized, double-blind, placebo-controlled, repeated measures study. The participants were assigned to a PA group (750 mg/day; Mediator®, Chemi Nutra, MN; n=7) or placebo group (PL; rice flower; n=7), delivered in capsule form that was identical in size, shape and color. Participants were tested for 1RM strength in the bench press (BP) and squat (SQ) exercise. Lean soft tissue mass (LM) was measured using dual-energy X-ray absorptiometry (DEXA). After base line testing, the participants began supplementing PA or PL for 8 weeks during a progressive resistance training program intended for muscular hypertrophy. Data was analyzed using magnitude-based inferences on mean changes for BP, SQ and LM. Furthermore, the magnitudes of the inter-relationships between changes in total training volume and LM were interpreted using Pearson correlation coefficients, which had uncertainty (90% confidence limits) of approximately ±0.25.

In the PA group, the relationship between changes in training volume and LM was large (r=0.69, ±0.27; 90% CL). However, in the PL group, the relationship was small (r=0.21, ±0.44; 90% CL). Changes in strength and LM in PA and PL groups, and qualitative inferences about the effects are recorded in Table 3-1.

TABLE 3-1

| Change in Measure | | | | Qualitative |
|---|---|---|---|---|
| | PA | PL | | Inference |
| | (mean + SD) | (mean + SD) | Difference | (+90% CL) |
| LM (Kg) | 1.7 ± 1.3 | 0.6 ± 1.5 | 1.1 ± 1.4 | Beneficial |
| SQ (Kg) | 17.2 ± 6.7 | 12.6 ± 6.1 | 4.5 ± 6.0 | Beneficial |
| BP (Kg) | 6.2 ± 6.1 | 3.6 ± 7.0 | 2.6 ± 6.2 | Likely |
| | | | | Beneficial |

In conclusion, PA supplementation was determined to beneficial at improving SQ and LM over PL by 26% and 64%, respectively. The strong relationship between changes in total training volume and LM in the PA group suggest that greater training volume can lead to the greater changes in LM. No such relationship was found with PL group. For the BP data, the PA group resulted in a 42% greater increase in strength over PL, although the effect is still under evaluation.

While more research is needed to elucidate the mechanism of action, the current findings indicate that in resistance trained men, supplementing 750 mg PA per day for 8 weeks provides greater changes in muscle mass and strength compared with resistance training alone.

### Example 4

### Lyso-Phosphatidic Acid Supplement Increases Lean Body Mass, Muscle Hypertrophy, Power and Strength Comparable to Whey Protein Following Resistance Exercise

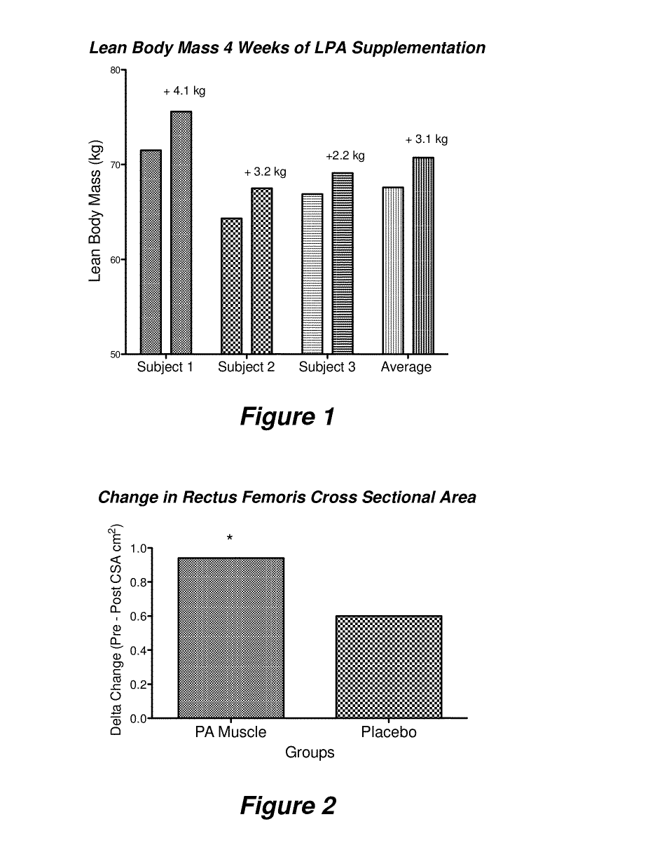

Lyso-phosphatidic acid (LPA) activates protein synthesis through the mTOR signaling pathway. Supplementing with LPA along with resistance training enhances lean body mass. Three highly resistance trained subjects with an average lean body mass of 66.9±3.6 kg trained for 4 weeks as a part of a daily undulating periodized resistance-training program centered around the following standard compound movements: the squat, the deadlift and the bench press. Subjects were supplemented with a 500 mg LPA formulation administered prior to their workouts and again after their exercise. Dual X-ray absorptiometry was used to determine changes in lean body mass. All 3 subjects increased in lean body mass an average of 3.1 kg. Individual subject data and average changes are illustrated in FIG. 1.

### Example 5

### Effects of Phosphatidic Acid Administration

The accretion of skeletal muscle tissue can be critical for a varied population including athletes and elderly. Skeletal muscle hypertrophy is largely mediated through increased muscle protein synthesis (MPS). The mammalian target of rapamycin (mTOR) has been recognized as the key protein complex through which MPS is regulated. Specifically elevations in energy status, amino acids, and growth factors have increased MPS through an mTOR dependent mechanism. One novel influence over mTOR is the phospholipid second messenger, phosphatidic acid. Previous research has suggested that phosphatidic acid up-regulates mTOR, but the research with phosphatidic acid as an oral supplement was inconclusive. This example provides a study investigating the effects of phosphatidic acid as an oral supplement on body composition, strength, and power. Indeed, phosphatidic acid supplementation was found to increase skeletal muscle hypertrophy, among other benefits.

The phenomenon of mechanotransduction has been revealed to operate through mTOR as well. During eccentric contraction, phospholipase D (PLD) is thought to be physically dislodged from the z-line of muscle tissue. This main enzyme, PLD, hydrolyzes phosphatidylcholine to yield phosphatidic acid (PA), a lipid second messenger, and choline. A significant elevation of intracellular PA was found to follow eccentric contractions in an ex vivo model. Furthermore, inhibition of the synthesis of PA by PLD also blocked the eccentric contraction induced increase in S6K1 phosphorylation, providing evidence that PA increases mTOR proliferation. Presently, PA is believed to activate mTOR through binding to the FKBP12 rapamycin binding (FRB) domain of mTOR.

Eccentric contraction induced endogenous production of PA is believed to elevate protein synthesis through an extracellular regulated kinase (ERK) independent mechanism. However, the effects of the provision of an exogenous source of PA were unknown. Research has demonstrated that exogenous addition of PA to rat fibroblasts increased PLD activity, PA content, and S6K1 phosphorylation all within the cell. Additionally, exogenous PA is hydrolyzed and then bound to endothelial differentiation gene (EDG), which subsequently increases ERK phosphorylation and PA content within the cell. Collectively, these findings indicate that exogenous provision of PA may up-regulate mechanisms responsible for augmenting MPS. Moreover, the mechanisms by which exogenous PA up-regulates are independent of the mechanisms attributed to endogenous PA production.

When considering a human model, exogenous elevations of PA may be provided through oral supplementation, while endogenous production could be fostered through a resistance training stimulus. Therefore, the combination of the two could result in greater skeletal muscle hypertrophy than resistance training alone. However, to date only one study has investigated the combination of oral PA supplementation combined with resistance training (RT). Specifically, Hoffman et al. (J. Int. Soc. Sports Nutr. 2012, 9:47) found that PA supplementation in humans undergoing progressive resistance training very likely resulted in greater increases in squat strength and lean mass over the placebo. However, this study was likely underpowered and subjects in the pilot study were not supervised. Finally, while the authors looked at indices of hypertrophy such as lean body mass, no direct measures of skeletal muscle hypertrophy were taken. Therefore, the results are inconclusive of whether or not PA supplementation specifically enhances skeletal muscle hypertrophy. The instant study investigates the effects of PA on skeletal muscle hypertrophy, strength, and power when consumed during a periodized RT program in a double-blind, placebo-controlled design to determine if PA supplementation leads to increased improvements in strength, skeletal muscle hypertrophy, and power.

Methods.

In summary, resistance-trained, male subjects were equally divided into experimental and control conditions, and each subject took part in an 8 week periodized resistance training program. The experimental condition (EXP) received 750 mg of phosphatidic acid, while the control condition (CON) received a visually identical placebo, in a double blind manner. Measurements of body composition, rectus femoris CSA, 1RM strength, and anaerobic power were taken prior to and following the 8 week training intervention. A 2×2 repeated measures ANOVA was used to determine group, time, and group×time interactions. A Tukey post-hoc was used to locate differences.

Experimental Approach. Participants were carefully matched according to their lean body mass (LBM), rectus femoris cross sectional area (CSA), and leg press 1-repetition-maximum (1RM,) and they were then equally divided into either the experimental (EXP) or control (CON) groups. All participants were required to abstain from consuming any muscle-building supplements for 1 month prior to pretest measures, be non-smokers, have RT experience of no less than one year, and have participated in RT at least three days per week for the past six months to be included in this study.

Measures of leg press and bench press 1RM, LBM, fat mass, total mass, and CSA were taken prior to and following the RT protocol. Ultrasonography measured muscle cross sectional area of the rectus femoris was used to determine hypertrophy. Dual X-ray Absorptiometery (DEXA) was used to determine changes in lean body mass (LBM) and fat mass, while the leg press and bench press were used to determine changes in strength.

Participants. Twenty-eight resistance-trained males (21.3±1.9 years) were recruited to participate in the study. Each participant reported themselves to be healthy and abstained from all muscle-building supplements for 1 month prior to beginning the investigation.

Resistance Training Protocol. Resistance training (RT) occurred three days per week, with 48-72 hours between RT sessions. Each body part was trained 1-2 times per week following a daily undulating periodized scheme. Each participant performed a 5 RM for each exercise that was performed during the first four weeks with the exception of the bench press and leg press in which true 1RM values were determined. These RM values were used to calculate the load used for each exercise for each participant. These exercises were altered to introduce a more novel stimulus. All participants were required to perform the prescribed number of repetitions with their prescribed weight. In the event that a subject reached muscular failure, a laboratory researcher assisted with the completion of the exercise. A comprehensive outline of the workouts is shown below in Table 5-1.

TABLE 5-1
Outline of Resistance Training Workouts.

| | M/W reps | Fri reps | M/W rest | Friday rest |
|---|---|---|---|---|
| week 1 | 12 | 5 | 45 s | 3-5 min |
| week 2 | 10 | 3 | 60 s | 3-5 min |
| week 3 | 8 | 2 | 90 s | 3-5 min |
| week 4 | 6 | 1 | 120 s | 3-5 min |
| week 5 | 12 | 5 | 60 s | 3-5 min |
| week 6 | 10 | 3 | 60 s | 3-5 min |
| week 7 | 8 | 2 | 90 s | 3-5 min |
| week 8 | 6 | 1 | 120 s | 3-5 min |

| | |
|---|---|
| Weeks 1-4 | Weeks 4-8 |

Monday

| | |
|---|---|
| leg press | leg press |
| leg extension | safety squat |
| leg curl | barbell lunge |
| hyperextension | SLD |
| bench press | bench press |
| incline dumbbell press | flat dumbbell press |
| close grip bench press | cable crossover |
| cable rope extensions | overhead triceps extensions |

Wednesday

| | |
|---|---|
| bent over row | pendlay row |
| barbell shrug | hexbar shrug |
| straight arm pulldown | pulldown |
| Australian row | decline dumbbell row |
| barbell shoulder press | dumbbell shoulder press |
| isolated barbell military | upright row |
| dumbbell lateral raise | barbell front raise |
| dumbbell bicep curls | barbell bicep curls |

Friday

| | |
|---|---|
| leg press | leg press |
| bench press | bench press |
| leg extension | safety squat |
| close grip bench press | flat dumbbell press |

Strength, Power, and Body Composition Testing. Strength was assessed via 1-RM testing of the leg press and bench press. Body composition (lean body mass, fat mass, and total mass) was determined on a Lunar Prodigy DXA apparatus (software version, enCORE 2008, Madison, Wis., U.S.A.). Skeletal muscle hypertrophy was determined via changes in ultrasonography determined CSA of the rectus femoris (General Electric Medical Systems, Milwaukee, Wis., USA). Power was assessed during a maximal cycling ergometry test. During the cycling test, the volunteer was instructed to cycle against a predetermined resistance (7.5% of body weight) as fast as possible for 10 seconds (Smith et al., J. Strength & Conditioning Research/National Strength & Conditioning Association 2001, 15:344-348). The saddle height was adjusted to the individual's height to produce a 5-10° knee flexion while the foot was in the low position of the central void. A standardized verbal stimulus was provided to the subjects. Power output was recorded in real time by a computer connected to the Monark standard cycle ergometer (Monark model 894e, Vansbro, Sweden) to during the 10-second sprint test. Peak power (PP) was recorded using Monark Anaerobic test software (Monark Anaerobic Wingate Software, Version 1.0, Monark, Vansbro, Sweden). From completion of Wingate tests performed over several days, interclass correlation coefficient for peak power was 0.96.

Diet Control and Supplementation. Two weeks prior to and throughout the study, subjects were placed on a diet consisting of 25% protein, 50% carbohydrates, and 25% fat by a registered dietician who specialized in sport nutrition. Subjects met as a group with the dietitian, and they were given individual meal plans at the beginning of the study. Daily total of calories were determined by the Harris Benedict equation and tracked by weekly logs to ensure compliance.

The EXP group received 750 mg of PA per day, while the CON group received 750 mg of rice flour, each delivered in 5 visually identical capsules. On RT days, participants consumed 450 mg of their respective supplement 30 minutes prior to RT and 300 mg immediately following RT with 24 grams of hydrolyzed collagen protein powder. On non-RT days, participants consumed 450 mg of their respective supplement with breakfast and the remaining 300 mg with dinner. Compliance was monitored by study managers.

Statistics. A repeated measures ANOVA model was used to measure group, time, and group by time interactions. If any main effects were observed, a Tukey post-hoc was employed to locate where differences occurred. All statistics were run using Statistica software (Statsoft, 2011).

Results.

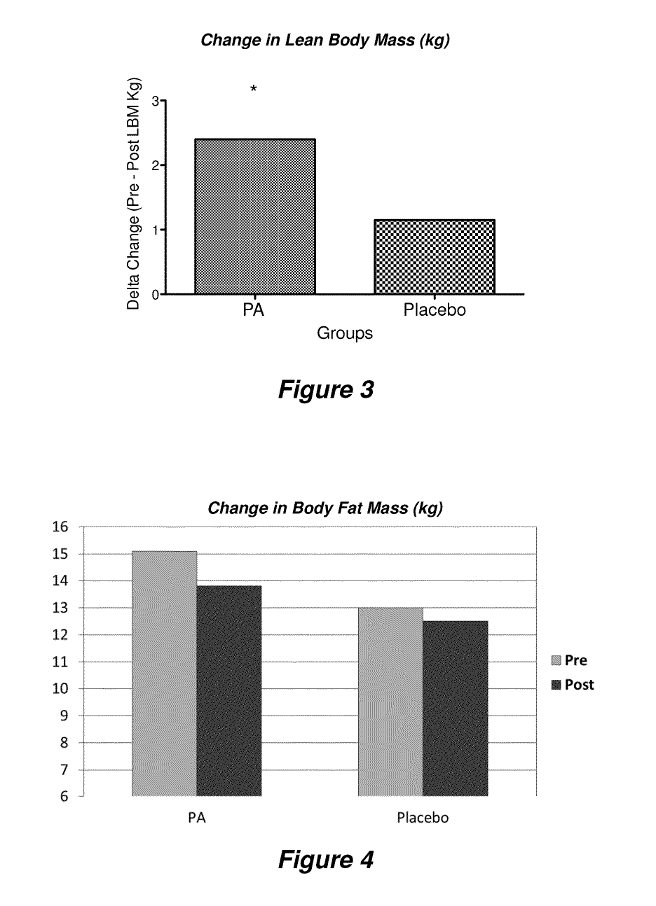

Body Composition. No differences existed between groups at baseline for any measure. There was a significant group×time effect (p=0.02) for cross sectional area (CSA) (FIG. 2), in which the EXP group increased (Effect Size (ES)=0.92) to a greater extent than the CON group (ES=0.52). There was a significant group×time effect (p=0.01) for lean body mass (LBM) (FIG. 3), in which the EXP group increased to a greater extent (ES=0.42) than the CON group (ES=0.26). Results with means and standard deviations are shown in Table 5-2.

TABLE 5-2
Resistance Training Results.

| | Placebo | PA | Effect Size |
|---|---|---|---|
| LP 1RM (lb.) | 498.9 ± 104.0 | 504.3 ± 109.1 | |
| PRE | | | |
| LP 1RM (lb.) | 570.4 ± 79.5* | 618.6 ± 79.7*^{#} | 0.78 vs. 1.2 |
| POST | | | |
| BP 1RM (lb.) | 201.4 ± 42.1 | 216.1 ± 29.8 | |
| PRE | | | |
| BP 1RM (lb.) | 211.8 ± 37.4* | 231.4 ± 27.3* | 0.25 vs. 0.5 |
| POST | | | |
| CSA (cm²) PRE | 4.5 ± 1.1 | 4.46 ± 1.1 | |
| CSA (cm²) POST | 5.1 ± 1.2* | 5.47 ± 1.3*^{#} | 0.52 vs. 0.91 |
| LBM (kg) PRE | 59.5 ± 4.7 | 59.7 ± 6.0 | |
| LBM (kg) POST | 60.7 ± 4.7* | 62.1 ± 5.5*^{#} | 0.26 vs. 0.42 |
| FAT (kg) PRE | 13.0 ± 6.5 | 15.1 ± 4.8 | |
| FAT (kg) POST | 12.5 ± 6.9* | 13.8 ± 4.2* | −0.07 vs. −0.28 |

*denotes significantly different from pre
^{#}denotes significantly different from placebo

There was a significant time effect (p=0.02) for Total Body Mass (TBM) in which the EXP group increased from 78.1±8.7 to 78.7±7.9 kg and the CON group increased from 75.7±5.8 to 76.5±6.1 kg, but no differences existed between groups (p=0.71). There was a significant time effect (p<0.01) for fat mass, in which there was a trend (P=0.068) for body fat mass to decrease to a greater extent in the EXP group (−0.28) than the CON group (−0.07), which decreased from 13.0±6.5 to 12.5±6.9 kg (FIG. 4).

Strength and Power. There was a significant group×time effect (p=0.04) for leg press 1RM, in which the EXP group increased to a greater extent (ES=1.2) than the CON group (ES=0.78). There was a significant time effect (p<0.01) for bench press 1RM, in which both the EXP (ES=0.5) and control groups (ES=0.25) increased; however, no differences were present between groups (p=0.11). There was a significant time effect (p<0.01) for Wingate peak power, which increased in the EXP group from 760.5±166.0 W to 822.8±217.3 W and from 733.5±105.8 to 797.3±122.3 W in the CON group; however, there were no differences between groups (p=0.97).

Discussion.

This study investigated the effects of PA supplementation on skeletal muscle hypertrophy, LBM, strength and power when consumed during a RT program in a double-blind, placebo-controlled design. The study determined that PA supplementation leads to increased improvements in skeletal muscle hypertrophy and strength. The results show that PA supplementation does improve strength and skeletal muscle hypertrophy over placebo, but does not significantly improve anaerobic power output. A unique finding of this study is the demonstration of a true improvement in muscle hypertrophy with PA supplementation as measured by muscle CSA.

Effects of Phosphatidic Acid Supplementation on Skeletal Muscle Hypertrophy and Lean Body Mass.

For decades, it has been well documented that resistance training (RT) leads to increases in muscle mass. However, clear mechanisms for muscle hypertrophy due to mechanical stimulus have only just begun to be elucidated. Research indicates that the lipid second messenger, PA, is at least partially responsible for translating the mechanical stimulus of RT into the chemical signal for skeletal muscle hypertrophy.

Endogenous production of PA leads to direct binding to and phosphorylation of the mTOR protein complex, subsequently resulting in increased activity of MPS markers. These effects appear to be independent of the PI3K and ERK signaling pathways. However, exogenous PA is hydrolyzed to lyso-PA, which binds to the G-protein-coupled receptor, EDG, on the cell membrane. The binding to EDG results in a cascade of events, which stimulate the ERK metabolic pathway and also increase PLD activity within the cell. The result is a dual mediated increase in mTOR signaling via increased PA content within the cell as well as activation of the ERK signaling pathway. Through the activation of multiple metabolic pathways, supplementing with PA can result in greater rates of MPS than RT alone and can explain the observed increase in skeletal muscle accretion and LBM when supplementing with PA.

While the results of Example 3 above found that changes in LBM were enhanced by PA supplementation, the effects were not as robust as the present study in either the control or experimental conditions. For example, the Example 3 results found no change in LBM in the placebo (+0.1 kg LBM), and found smaller effects in the experimental (+1.7 kg LBM). These findings indicate that the training stimulus was inadequate to enhance LBM by itself, and that changes in LBM were primarily driven by the supplement itself. The findings were the result of non-supervised training; research demonstrates greater increases in neuromuscular adaptations in supervised (this example) as compared to non-supervised training (Example 3). In contrast, the present study found increases in both LBM and hypertrophy in both the placebo and PA conditions due to the supervised resistance training. These findings indicate that skeletal muscle hypertrophy was driven by both endogenous (training) and exogenous (PA supplementation) mechanisms.

Past research strongly suggests that resistance training alone does not provide a strong stimulus for fat loss (Wilson et al., J. Strength & Conditioning Research/National Strength & Conditioning Association 2012, 26:2293-2307). In the current study, a clear trend was observed (p=0.068) for PA to decrease body fat (ES=0.28), which is remarkably significant due to the placebo controlled double blind nature of the study. Thus, PA-driven changes in protein synthesis can increase substrate utilization to a point that activates key regulators of fat metabolism such as AMPK.

Strength and Power. Strength is one of the most critical attributes underlying success in sport. The collective results of the present study, as well as those from Example 3, indicate that changes in strength following supervised and non-supervised resistance training are enhanced. The observed increases in skeletal muscle CSA contribute to the increases observed in strength. As no mechanism for PA currently exists that would propose a direct implication for strength, the increases in strength are therefore due to the associated increases in CSA. No differences were observed for power likely because the participants did not train with a power-oriented stimulus. Rather, they trained for hypertrophy twice per week, and strength once per week.

Conclusions. This is the first study to show that oral PA supplementation directly augments changes in skeletal muscle hypertrophy following a chronic RT stimulus. Benefits for increases in lean body mass and strength were also confirmed. PA supplementation resulted in a significant increase in leg press strength compared with placebo (group×time, p<0.05). In addition, this research shows that there was a trend in the PA group (p=0.06) for greater declines in fat mass compared to the placebo.

In summary, PA supplementation resulted in a 60% greater increase in 1RM leg press strength (lbs), a 47% greater increase in 1RM bench press strength (lbs), a 68% greater increase in femoral muscle CSA (cm²), a 200% greater increase in lean body mass (kg), and a 260% decrease in fat mass (kg) compared to placebo. Thus, for the first time, experimental results show that PA supplementation augments adaptations in skeletal muscle hypertrophy in resistance trained individuals in combination with a reduction in body fat. Moreover, PA supplementation provides increases of strength and lean body mass.

### Example 6

### Safety of Soy-Derived Phosphatidic Acid Supplementation

The mammalian target of rapamycin (mTOR) has been shown to regulate rates of muscle protein synthesis. One novel nutritional activator of mTOR is the phospholipid Phosphatidic Acid (PA). We have recently found that PA supplementation over 8 weeks of resistance training augmented responses in skeletal muscle hypertrophy and strength. However, we are unaware of research investigating the safety of PA in human subjects. This study investigated the effects of 8 weeks of 750 mg per day of PA supplementation on safety parameters in healthy college aged males.

Twenty-eight healthy, college aged male subjects participated in the study. Subjects were equally divided into experimental and control conditions. The experimental condition (EXP) received 750 mg of soy-derived PA (Mediator™, Chemi Nutra, White Bear Lake, Minn.), while the control condition (CON) received a visually identical placebo (rice flour). Measures of cardiovascular, kidney, and liver function were analyzed with a full CMP and CBC which included: total, high density, and low density lipoproteins, blood glucose, blood urea nitrogen, creatinine, eGFR, Na, K, Cl, CO₂, Ca, protein, albumin, globulin, albumin:globulin ratio, total bilirubin, alkaline phosphatase, aspartate aminotransferase, and alanine aminotransferase. In addition, a sample of urine was submitted for analysis. A 2×2 repeated measures ANOVA was used to determine group, time, and group×time interactions. A Tukey post-hoc was used to locate differences.

No differences were found at baseline in blood chemistry and hematology between the CON and EXP supplemented groups. Additionally no differences were observed in urinalysis values between the groups. Moreover no group or group×time effects were found following 8 weeks of supplementation.

Accordingly, soy-derived PA was determined to be a safe nutritional supplement for healthy college aged subjects if taken up to a dosage of 750 mg over an eight week period.

### Example 7

### Effects of G3P and Chemi Nutra PA on mTOR Signaling in C2C12 Myoblasts

In this study, C2C12 myoblasts were incubated with 10 μM or 30 μM of various lipids for 20 minutes. The cells were then analyzed for changes in p70(389) phosphorylation as a marker of mTOR signaling.

P16 C2C12 myoblasts were plated at approximately 20% confluence and grown for 36 hour in 10% FBS High Glucose DMEM. Cells were switched to 2 mL/well serum free High Glucose DMEM (no antibiotics) for 16 hours prior to the experiment. Cells were 80% confluent at the time of the experiment.

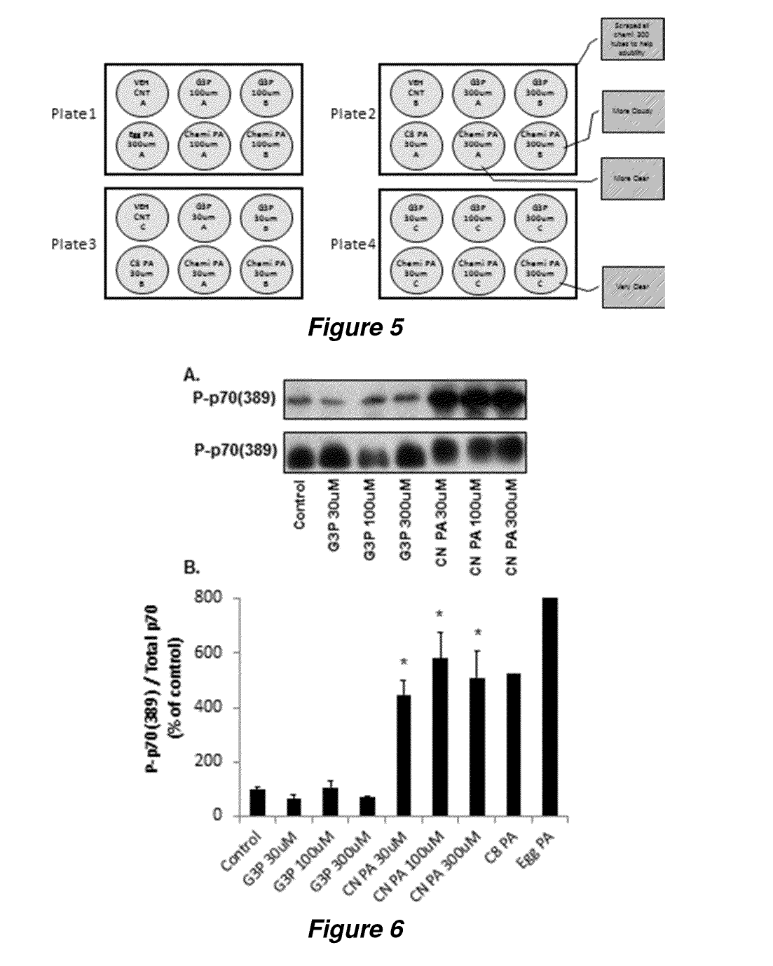

Cells were then stimulated with 30 μM of C8 PA, 300 μM of Egg PA, 30-300 μM of Glycerol 3-Phosphate or 30-300 μM of Chemi Nutra PA. This was added to the serum free media as detailed on the next page and the cells were then incubated for 20 min and collected in 150 μL WIK buffer+inhibitors. See FIG. 5.

The assays were performed as follows.

Day 1. Plate cells on 6 well dishes at apx 20% and grow in 10% FBS-DMEM+antibiotics.

Day 2. Prep all tubes for sample collection and stimulations. Serum starve with DMEM—no antibiotic media. Plate 1 at 8:30 PM, Plate 2 at 8:40 PM, Plate 3 at 9:10 PM, Plate 4 at 9:20 PM.

Day 3. Prep G3P stock stimulant solutions, setup PBS solutions. Thaw inhibitors, Bring C8, Egg PA, Chemi Nutra PA up to room temp. Set up for collection, set up for Cell Culture Conditions (pipettes, tips, stimulant solutions, etc.).

### Dry and Prep 2×100 uM Chemi PA and 1×300 uM Egg PA for Plate 1. Stimulate Plate 1.

### Dry and Prep 2×300 uM Chemi PA and 1×30 uM C8 PA for Plate 2. Stimulate Plate 2

Prep Fresh WIK Buffer with Inhibitors (Enough for 2 Plates)
124.8 μL of β-Glycerophosphate (600 mM stock)
75 μL of NaF (1M stock)
15 μL of PMSF (0.2 M stock)
3 μL of Leupeptin (10 mg/mL stock)
3 μL of Na₃VO₄ (1 M stock)
2.8 mL of WIK Buffer stock (ICE COLD)

### Collect Plate 1 in 150 ul WIK+Inhibitors/well.

### Collect Plate 2 in 150 ul WIK+Inhibitors/well.

### Dry and Prep 2×300 uM Chemi PA and 1×30 uM C8 PA for Plate 3. Stimulate Plate 3.

### Dry and Prep 30 um, 100 um and 300 uM Chemi PA for Plate 4. Stimulate Plate 4.

Prep Fresh WIK Buffer with Inhibitors (Enough for 2 Plates) as described above.

### Collect Plate 3 in 150 ul WIK+Inhibitors/well.

### Collect Plate 4 in 150 ul WIK+Inhibitors/well.

Stimulant Prep Instructions.

Vehicle Control.

All stimulant conditions will use 100 μL of PBS as the vehicle, so for all control wells incubate with 100 μL of PBS only (aliquot taken from same bottle used in stimulant preps).

Chemi Nutra PA.

The original stock powder (−50% pure PA) was diluted at 20 mg/mL in chloroform yielding a solution at approximately 10 mg/mL of PA.

Chemi Nutra PA 300 μM.

Stimulate with 300 μM Chemi Nutra PA using 100 μL of solution. The media volume is 2 mL, so add 0.6 μmol of PA to the 2 ml of media (300 μmol/L=0.300 μmol/mL). Make 125 μL of solution, thus use 0.75 μmol of PA in 125 μL of PBS.

Chemi Nutra PA MW=696.92 g/mol=696.92 μg/μmol*0.75 μmol=533.7 μg=0.5337 mg (stock=10 mg/mL, so need 53.37 μL of stock). Dry the stock under nitrogen and re-suspend in 125 μL of dPBS with 3 min of sonication and repeated vortexing. Immediately use for the cell stimulation.

Chemi Nutra PA 100 μM.

Stimulate with 100 μM Chemi Nutra PA using 100 μL of solution. The media volume is 2 mL, so add 0.2 μmol of PA to the 2 mL of media (100 μmol/L=0.100 μmol/mL). Make 125 μL of solution, thus use 0.25 μmol of PA in 125 μL of PBS.

Chemi Nutra PA MW=696.92 g/mol=696.92 μg/μmol*0.25 μmol=177.9 μg=0.1779 mg (stock=10 mg/mL, so need 17.79 μL of stock). Dry the stock under nitrogen and re-suspend in 125 μL of dPBS with 3 minutes of sonication and repeated vortexing. Immediately use for the cell stimulation.

Chemi Nutra PA 30 μM.

Stimulate with 30 μM Chemi Nutra PA using 100 μL of solution. The media volume is 2 mL, so add 0.06 μmol of PA to the 2 mL of media (30 μmol/L=0.030 μmol/mL). Make 125 μL of solution, thus prepare 0.075 μmol of PA in 125 μL of PBS.

Chemi Nutra PA MW=696.92 g/mol=696.92 μg/μmol*0.075 μmol=52.26 μg=0.0523 mg (stock=10 mg/mL, so prepare 5.23 μL of stock). the stock under nitrogen and re-suspend in 125 μL of dPBS with 3 min of sonication and repeated vortexing. Immediately use for the cell stimulation.

Egg PA 300 μM.

Stimulate with 300 μM Egg PA using 100 μL of solution. The media volume is 2 mL, so add 0.6 μmol of PA to the 2 mL of media (300 μmol/L=0.300 μmol/mL). Make 125 μL of solution, thus use 0.75 μmol of PA in 125 μL of PBS.

Egg PA MW=696.92 g/mol=696.92 μg/μmol*0.75 μmol=533.7 μg=0.5337 mg (stock=10 mg/mL, so prepare 53.37 μL of stock). Dry the stock under nitrogen and re-suspend in 125 μL of dPBS with 3 min of sonication and repeated vortexing. Immediately use for the cell stimulation.

C8 PA.

Stimulate with 30 μM C8 PA using 100 μL of solution. The media volume is 2 mL, so add 0.06 μmol of PA to the 2 mL of media (30 μmol/L=0.030 μmol/mL). Make 125 μL of solution, thus prepare 0.075 μmol of PA in 125 μL of PBS.

C8 PA MW=446.45 g/mol=446.45 μg/μmol*0.075 μmol=33.48 μg=0.03348 mg (stock=10 mg/mL, so prepare 3.35 μL of stock). Dry the stock under nitrogen and re-suspend in 125 μL of dPBS with 3 min of sonication and repeated vortexing. Immediately use for the cell stimulation.

α-Glycerophosphate (G3P).

A stock powder of disodium glycerophosphate (G3P) was obtained from TCI (catalog #G0096) and was used to make a 6 mM stock solution in PBS. G3P MW=216.04 g/mol. Make 45 ml of a 6 mmol/L solution=6 μmol/ml*45 ml=270 mmol. 216.04 ug/μmol*270 μmol=58320 μg=58.3 mg dissolved in 45 mL PBS. This stock solution was made fresh on the day of the experiment.

G3P 300 μM.

Stimulate with 300 μM G3P using 100 μL of solution. The media volume is 2 mL, so add 0.6 μmol of G3P to the 2 mL of media (300 μmol/L=0.300 μmol/mL). Prepare stock solution with 6 mmol/L=6 μmol/mL. Adding 0.1 mL of the 6 mM stock solution to the media is equivalent to adding 0.6 μmol.

G3P 100 μM.

Stimulate with 100 μM G3P using 100 μL of solution. The media volume is 2 mL, so add 0.2 μmol of G3P to the 2 mL of media (100 μmol/L=0.100 μmol/mL). Prepare stock solution with 6 mmol/L=6 μmol/mL. Dilute the stock solution to 2 mM by combining 333 uL of 6 mM solution plus 667 uL of PBS. Adding 0.1 mL of the 2 mM stock solution to the media is equivalent to adding 0.2 μmol.

G3P 30 μM.

Stimulate with 30 μM G3P using 100 μL of solution. The media volume is 2 mL, so add 0.06 μmol of PA to the 2 mL of media (30 μmol/L=0.030 μmol/mL). Stock solution with 6 mmol/L=6 μmol/mL. Dilute the stock solution to 0.6 mM by combining 100 uL of 6 mM solution plus 900 uL of PBS. Adding 0.1 mL of the 0.6 mM stock solution to the media is equivalent to adding 0.06 μmol.

FIG. 6 shows that the Chemi Nutra PA activates mTOR signaling. C₂C₁₂ myoblasts were stimulated with various dose of glucose-3-phosphoate (G3P), Chemi Nutra PA (CN PA) or the vehicle (control) for 20 minutes as described on the previous above. Stimulations with C8 PA or Egg PA were used as positive controls. A. Western blot of p70 phosphorylated on the threonine 389 residue [P-p70(389)] was compared to total p70 and used as a marker of mTOR signaling. B. Graphical representation of the P-p70(389) to total p70 ratio expressed as a percent of the control values. * P<0.05 compared to control.

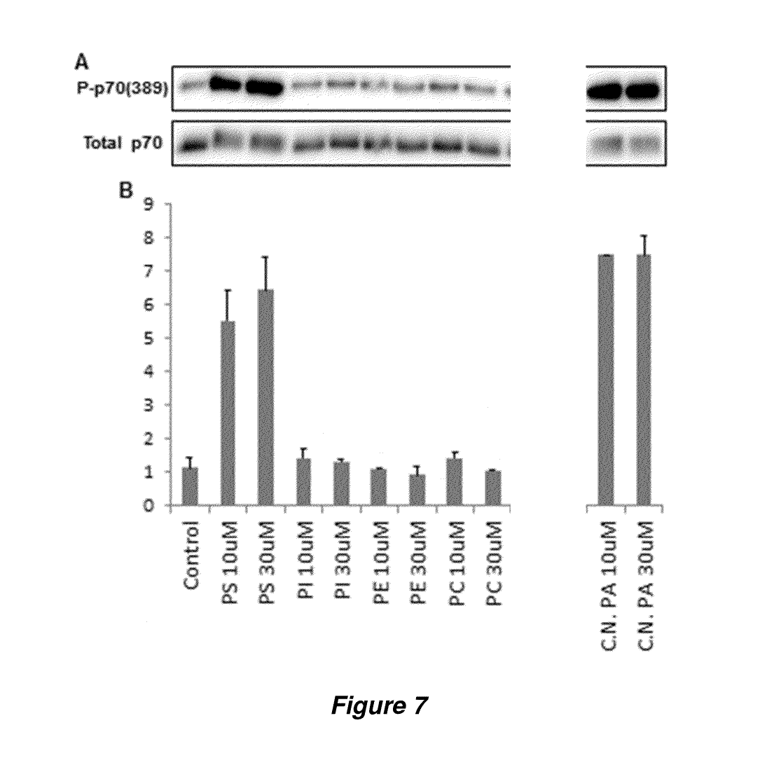

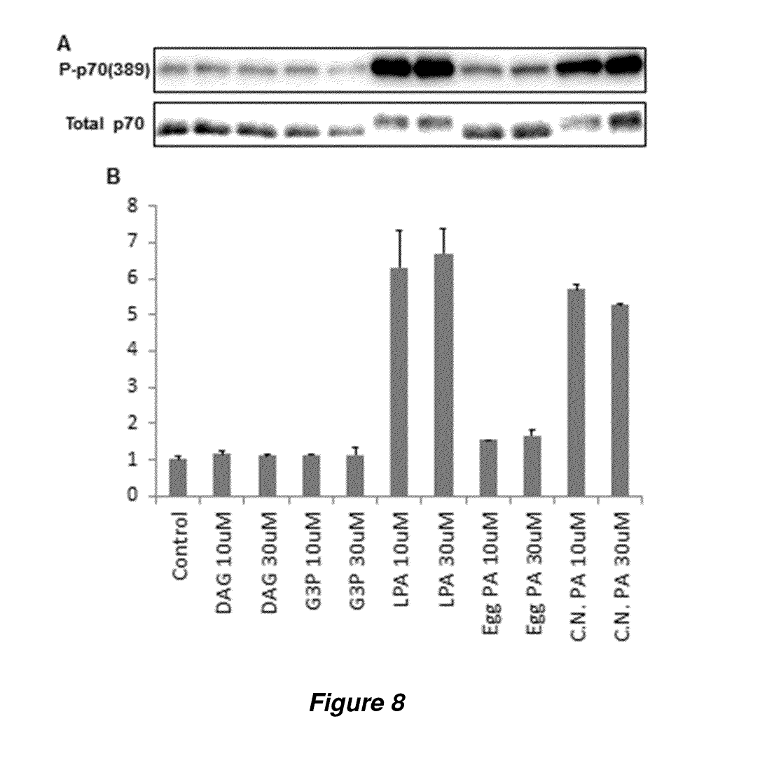

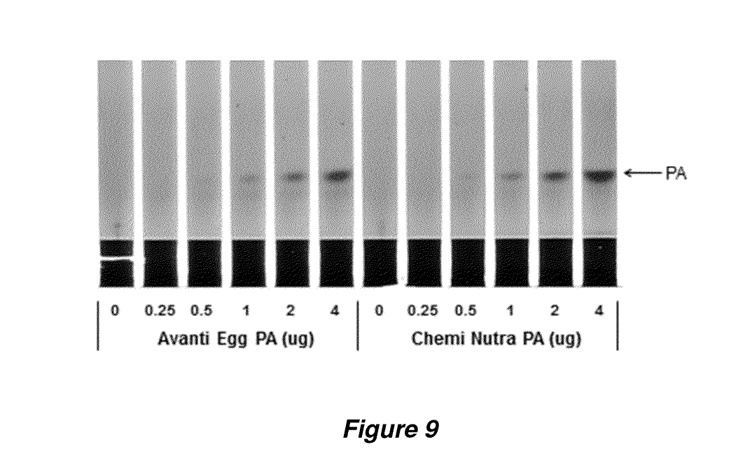

Further studies using similar methods were carried out to compare PC, PI, PE, PS, LPA, G3P, egg-PA and DAG to the Chemi Nutri PA. Results are illustrated in FIGS. 7-9. Accordingly, soy-derived PA, egg-derived PA, soy-derived PS, and soy-derived lyso-PA are each activators of mTOR, and as such, can be used in the methods of the invention, for example, in place of PA, to provide the described beneficial effects upon dietary supplementation with effective amounts. Additionally, soy-PA was found to be a robust activator of mTOR, thus can be used to effect the beneficial results with lower dosages with greater results.

### Example 8

### Analysis of Lyso-Phosphatidic Acid and Phosphatidic Acid in Human Plasma

Lyso-phosphatidic acid (LPA) and phosphatidic acid (PA) from human plasma includes a physical distribution of molecules varying in their fatty acids esterified to the sn-1 and sn-2 positions of glycerol-3-phosphate. The differences in polarity among the fatty acid substituted structures provide a means to resolve individual species under reversed phase chromatographic conditions. This analysis coupled with selective mass spectrometric detection based on molecular weight and distinctive fragmentation for LPA and PA compounds provided the basis for quantitation in the samples described herein. Total LPA and PA content are measured as the sum of the individual species measured.

Human plasma of a subject was analyzed after administration of lyso-phosphatidic acid and phosphatidic acid. One subject consumed 2 g of PA in form of Mediator® (Chemi Nutra). The data from the study provided the composition and concentration of PA and lyso-PA in human blood plasma as a baseline value, and changes in PA and lyso-PA concentrations after 30 minutes, 1 hour, 2 hours, 3 hours and 7 hours. It was determined that PA administration results in increases in lyso-PA and PA concentrations in human blood plasma.

An initial proof of principle analysis was performed on a NIST certified, 1950 Normal Human Plasma control (1950 Metabolites in Human Plasma; National Institute of Standards and Technology, Technology Administration, U.S. Department of Commerce), which is known to contain phospholipid compounds. This PA compound-containing control plasma has been quantified by mass spectrometry for multiple diacyl PA compounds (Dennis et. al., J. Lipid Res. 2010 November; 51(11): 3299-3305). Confirmation of the reported molecular species was performed under the reversed phase liquid chromatography/mass spectrometry (LC/MS) method reported herein.

LC/NIS/NIS Method.

An ultra performance liquid chromatograph with triple quadrupole mass spectrometry (LC/MS/MS) method was utilized to measure the content of LPA and PA molecular species in human plasma. A Waters Acquity™ Ultra-performance liquid chromatography system coupled with an AB Sciex 5500 tandem quadrupole mass spectrometer was the instrumentation platform.

LC/MS/MS conditions were as follows.

Column: Agilent, Eclipse™ XDB-C8 4.6×150 mm, 1.8 μm, sn. USHAN01857, pn. 927975-906.
Flow=1 mL/minute at 40° C.
Mobile phases: A=70:28.5:1.5 (/v) methanol:water:acetic acid with 0.08% TEA/v;

B=98.5:1.5 (/v) methanol:acetic acid with 0.08% TEA/v.

### Gradient:

| Time (min) | A % | B % |
|---|---|---|
| 0.0 | 40 | 60 |
| 1.0 | 40 | 60 |
| 3.0 | 0 | 100 |
| 5.5 | 0 | 100 |
| 5.51 | 40 | 60 |
| 7.5 | 40 | 60 |

The global MS parameter settings used for the electrospray source were:

| | |
|---|---|
| Curtain gas | 20 |
| Collision gas | 7 |
| Ion spray voltage | −4500 V |
| GS1 | 50 |
| GS2 | 50 |

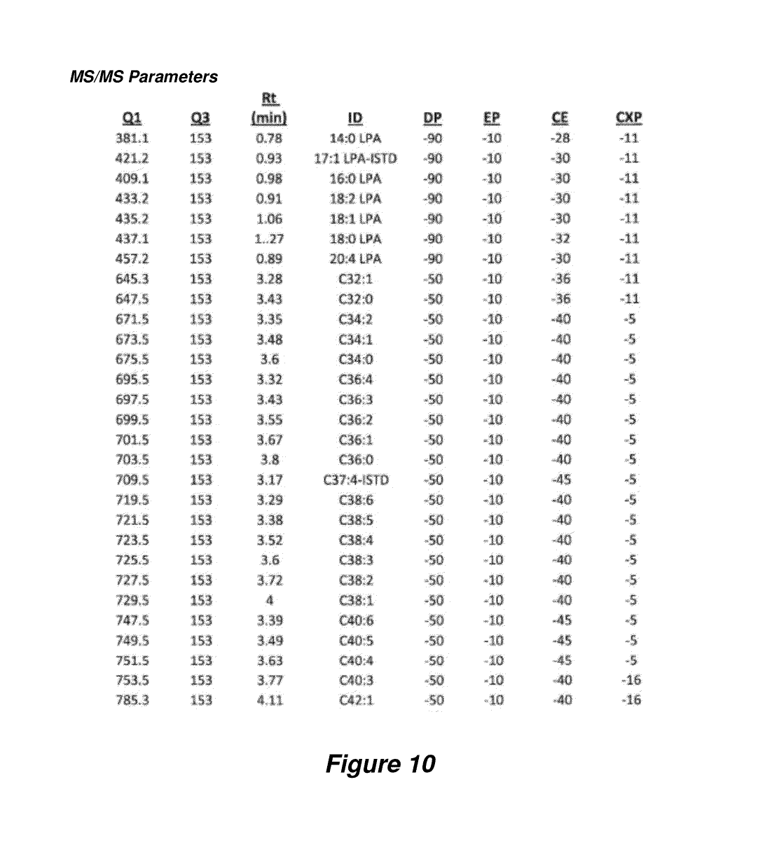

The identified molecular species were detected using a multiple reaction monitoring (MRM) technique for the -ve mode exact mass of [M-H]⁻ and the corresponding fragment 153 amu. The MS settings for the PA molecular species are provided in FIG. 10.

Sample Preparation.

The plasma samples and an NIST 1950 Human Plasma control were extracted with internal standards spiked into each. The internal standards were added as a mixture of 17:1 LPA at 5 nM and 17:0-20:4 PA (37:4 PA) at 50 nM in methanol. The extraction procedure was performed using HPLC grade solvents. The following procedure was carried out for each sample.

1. Add 200 μL of plasma to a 1.5 mL Eppendorf tube and 1.0 mL of the internal standard solution.

2. Vortex mix each sample 15 seconds to fully homogenize the plasma.

3. Place in −20° C. freezer for 20 minutes.

4. Centrifuge at 14,000 rpm at room temperature for 5 minutes.

5. Decant methanol from pellet into respective 16×10 mm screw cap test tubes.

6. Add 1.0 mL of water and mix.

7. Add 1.0 mL of chloroform and vortex mix for 1 min (3×20 sec).

8. Centrifuge at 3600 rpm at room temperature (−22° C.) for 10 minutes.

9. Remove lower layer to a separate 16×100 mm screw cap tube.

10. Repeat steps 7-9 combining the respective lower (chloroform) layers.

11. Evaporate the solvent from each under nitrogen gas stream at room temperature.
12. Re-dissolve the residues with 200 μL of methanol.

13. Transfer each to 1.8 mL injection vials with a 500 μL insert inside for assay by LC/MS/MS.

Calculations.

The content of each molecular species was calculated from the internal standards present in each sample extract. The 17:1 LPA response was used for LPA compounds and 37:4 PA response was used for PA compounds within the linear range of the method, approximately 1 nM-10 μM. The equations for LP A and P A content are:

$$LPA ( nM ) = \frac{Area of LPA peak}{Area 17 \text{:} 1 LPA peak} \times \frac{5 nM \times 1.0 mL added}{0.2 mL final volume}$$

$$PA ( nM ) = \frac{Area of PA peak}{Area 37 \text{:} 4 PA peak} \times \frac{50 nM \times 1.0 mL added}{0.2 mL final volume}$$

Each analyte concentration was corrected for background through subtraction of corresponding MRM signal from an internal standard only solution.

Quality Control.

The NIST 1950 Human Plasma control is reported to contain 2.5 μM PA as a sum of individual molecular species. Assay under the method above reported 0.69 μM LPA with 2.04 μM PA.

Plasma Sample Results.

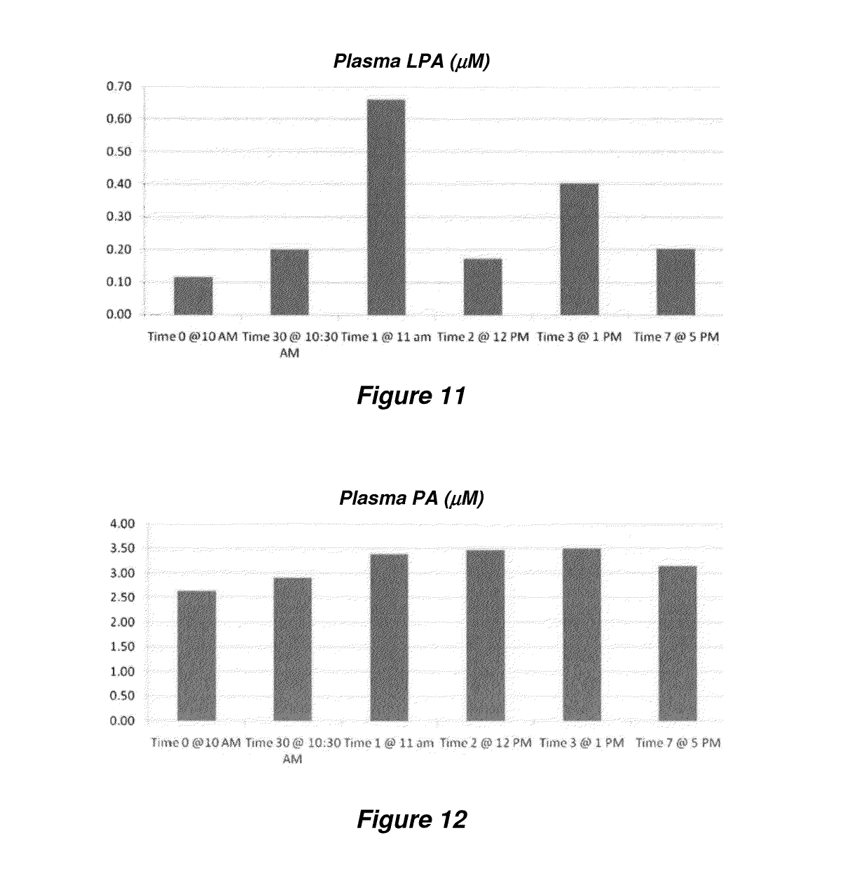

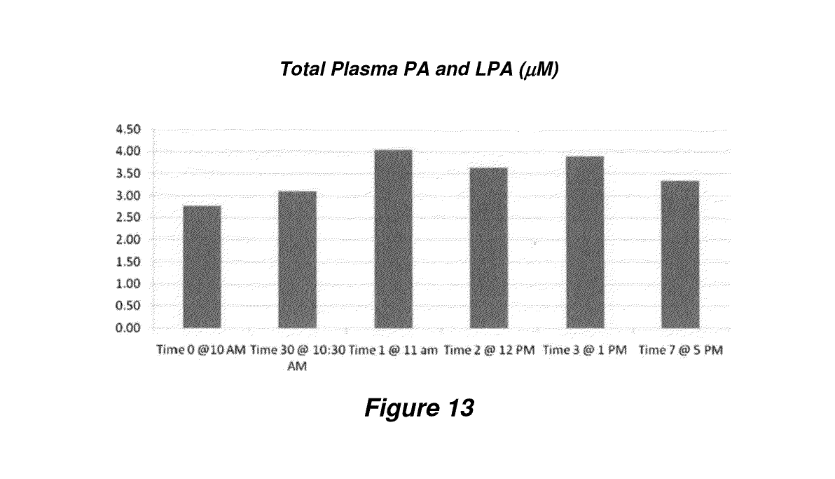

The LPA and PA plasma concentration are reported and compared in the Table 8-1 and FIGS. 11-13.

TABLE 8-1
PA Comparison (nM) background corrected.

| | Time 0 @10 | Time 30 @ | Time 1 @ 11 | Time 2 @ 12 | Time 3 @ 1 | Time 7 @ 5 |
|---|---|---|---|---|---|---|
| Cmpd | AM | 10:30 AM | am | PM | PM | PM |
| 14:0 LPA | 13.00 | 22.92 | 15.97 | 10.47 | 30.58 | 18.54 |
| 17:1 LPA-ISTD | 0.00 | 0.00 | 0.00 | 0.00 | 0.00 | 0.00 |
| 16:0 LPA | 51.33 | 74.64 | 68.94 | 65.04 | 156.39 | 91.92 |
| 18:2 LPA | 21.64 | 30.09 | 49.43 | 24.89 | 35.32 | 35.94 |
| 18:1 LPA | 8.84 | 22.40 | 31.30 | 13.22 | 22.24 | 17.40 |
| 18:0 LPA | 0.00 | 2.88 | 12.53 | 5.04 | 7.37 | 4.64 |
| 20:4 LPA | 20.07 | 47.44 | 483.48 | 52.89 | 152.75 | 34.17 |
| C32:1 | 38.60 | 44.34 | 40.48 | 45.41 | 34.12 | 28.27 |
| C32:0 | 3.78 | 17.28 | 7.26 | 13.74 | 25.73 | 7.58 |
| C34:2 | 410.09 | 405.53 | 734.06 | 483.22 | 510.38 | 522.25 |
| C34:1 | 196.56 | 199.23 | 250.43 | 329.36 | 293.63 | 352.06 |
| C34:0 | 5.53 | 3.60 | 2.73 | 7.19 | 1.86 | 4.30 |
| C36:4 | 271.16 | 267.90 | 388.63 | 353.79 | 303.66 | 322.38 |
| C36:3 | 267.39 | 210.86 | 185.37 | 202.65 | 293.26 | 200.65 |
| C36:2 | 212.50 | 234.00 | 303.55 | 500.16 | 351.46 | 251.73 |
| C36:1 | 92.81 | 82.65 | 82.47 | 99.83 | 76.88 | 99.04 |
| C36:0 | 0.19 | 0.00 | 0.00 | 0.00 | 0.00 | 0.00 |
| C37:4-ISTD | 0.00 | 0.00 | 0.00 | 0.00 | 0.00 | 0.00 |
| C38:6 | 95.94 | 158.76 | 175.07 | 149.83 | 234.12 | 188.86 |
| C38:5 | 52.08 | 66.49 | 79.07 | 117.97 | 77.05 | 100.57 |
| C38:4 | 259.68 | 299.07 | 320.45 | 347.66 | 400.65 | 257.38 |
| C38:3 | 80.62 | 118.37 | 75.92 | 108.77 | 106.70 | 101.30 |
| C38:2 | 12.59 | 13.06 | 13.79 | 11.96 | 11.01 | 11.15 |
| C38:1 | 0.00 | 0.00 | 0.00 | 0.00 | 0.00 | 0.00 |
| C40:6 | 124.41 | 155.37 | 144.22 | 104.86 | 187.25 | 150.31 |
| C40:5 | 232.39 | 302.26 | 253.46 | 301.91 | 247.51 | 283.88 |
| C40:4 | 288.55 | 333.51 | 328.65 | 293.12 | 330.24 | 255.76 |
| C40:3 | 13.48 | 9.47 | 8.13 | 11.04 | 16.86 | 15.22 |
| C42:1 | 0.00 | 0.00 | 0.00 | 0.00 | 0.00 | 0.00 |
| LPA (uM) | 0.11 | 0.20 | 0.66 | 0.17 | 0.40 | 0.20 |
| PA (uM) | 2.66 | 2.92 | 3.39 | 3.48 | 3.50 | 3.15 |
| Total (uM) | 2.77 | 3.12 | 4.06 | 3.65 | 3.91 | 3.36 |

While soy-derived PA does not contain any arachidonic acid (20:4); nevertheless, PA administration resulted in a 24 fold increase in 20:4-lyso-PA. Accordingly, after administration of soy-PA, the soy-PA is converted to G3P during absorption and the body attaches 20:4 to G3P to yield 20:4-lyso-PA, which was unexpected. Therefore, administration of PA provides a method to increase 20:4-lyso-PA by administering soy-PA. Additionally, lyso-PA showed an unusual absorption kinetic, first a sharp increase from baseline, then a sharp drop, followed by another increase. Potentially, lyso-PA absorption increases as the Mediator PA is absorbed as lyso-PA with lower amounts also absorbed as PA, where lyso-PA is metabolized quickly with a concomitant drop in plasma concentration, followed by an increase as part of the PA is converted into lyso-PA.

The formation of 20:4-lyso-PA, most likely from G3P, supports that administering G3P can provide an increase in lyso-PA and PA plasma concentrations. 20:4-lyso-PA can only be created if both fatty acids are cleaved from PA during absorption, which indicates that when G3P is administered, plasma lyso-PA concentrations are increased as well. Even if G3P is not a direct mTOR activator, G3P can be converted to lyso-PA or PA during absorption. Accordingly, G3P acts as a form of pro-drug for lyso-PA and PA.

### Example 9

### Dosage Forms

The following formulations illustrate representative dosage forms that may be used for the therapeutic or prophylactic administration of a compound (e.g., PA) described herein, a compound or composition specifically disclosed herein (e.g., a composition that includes a form of PA or a phospholipid described herein), or a pharmaceutically acceptable salt or solvate thereof (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Tablet 3 | mg/tablet |
|---|---|
| ‘Composition X’ | 400 |
| Rice flour | 400 |
| Magnesium stearate | 50 |
| | 850 |

These formulations may be prepared by conventional procedures well known in the pharmaceutical art. It will be appreciated that the above compositions may be varied according to well-known techniques to accommodate differing amounts and types of active ingredient ‘Composition X’ (e.g., PA and/or other actives described herein). Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for increasing skeletal muscle hypertrophy comprising orally administering to a subject an effective amount of phosphatidic acid on a daily basis for at least two weeks, wherein the subject participates in resistance training during the phosphatidic acid administration, resulting in increased skeletal muscle hypertrophy in the subject.

2. The method of claim 1 wherein the increased skeletal muscle hypertrophy is accompanied by an increase in skeletal muscle strength.

3. The method of claim 1 wherein the increased skeletal muscle hypertrophy comprises an increase in muscle hypertrophy of the rectus femoris muscles.

4. The method of claim 1 wherein the increased skeletal muscle hypertrophy is accompanied by an increase in lean body mass.

5. The method of claim 4 wherein the increased skeletal muscle hypertrophy is accompanied by a decrease in body fat composition.

6. The method of claim 1 wherein the effective amount of phosphatidic acid is about 100 mg per day to about 1000 mg per day.

7. The method of claim 5 wherein the effective amount of phosphatidic acid is about 375 mg per day to about 750 mg per day.

8. The method of claim 7 wherein the effective amount of phosphatidic acid is about 500 to about 750 mg per day.

9. The method of claim 1 wherein administering phosphatidic acid to the subject is carried out on a daily basis for at least eight weeks.

10. The method of claim 1 wherein the resistance training during the phosphatidic acid administration is carried out for at least eight weeks.

11. A method of reducing body fat composition in a subject comprising orally administering to a subject an effective amount of phosphatidic acid on a daily basis for at least two weeks, wherein the subject participates in resistance training during the phosphatidic acid administration, resulting in decreased body fat in the subject.

12. The method of claim 11 wherein the decreased body fat in the subject is accompanied by an increase in skeletal muscle hypertrophy.

13. The method of claim 12 wherein the increased skeletal muscle hypertrophy comprises an increase in muscle hypertrophy of the rectus femoris muscles.

14. The method of claim 11 wherein the decreased body fat in the subject is accompanied by an increase in lean body mass.

15. The method of claim 11 wherein administering phosphatidic acid to the subject is carried out on a daily basis for at least eight weeks.

16. The method of claim 15 wherein the resistance training during the phosphatidic acid administration is carried out for at least eight weeks.

17. The method of claim 16 wherein the decrease in body fat in the subject is a decrease of at least 0.5% body fat.

18. The method of claim 16 wherein the decrease in body fat in the subject is a decrease of at least 1% body fat.

19. The method of claim 18 wherein the effective amount of phosphatidic acid is about 100 mg per day to about 1000 mg per day.

20. The method of claim 19 wherein the effective amount of phosphatidic acid is about 375 to about 750 mg per day.
